(19) United States
(12) Patent Application Publication
(10) Pub. No.: US 2019/0316067 A1
(43) Pub. Date: Oct. 17, 2019

(54) PHOTO-BIOREACTOR DEVICE AND METHODS

(71) Applicant: ARBOREA LTD, Surrey (GB)

(72) Inventor: Julian Paul MELCHIORRI, Surrey (GB)

(73) Assignee: ARBOREA LTD, Surrey (GB)

(21) Appl. No.: 16/465,648

(22) PCT Filed: Dec. 1, 2017
(86) PCT No.: PCT/GB2017/053642

§ 371 Date: May 31, 2019

(30) Foreign Application Priority Data
Dec. 1, 2016 (GB) PCT/GB2016/053786
Jun. 5, 2017 (GB) 1708940.0

(51) Int. Cl. C12M 1/00; C12M 1/04; C12M 3/00; C12M 1/12

(52) U.S. Cl. CPC C12M 21/02; C12M 23/20; C12M 23/22; C12M 23/24; C12M 31/10; C12M 25/02; C12M 25/06; C12M 27/20; C12M 23/46

(57) ABSTRACT

Photobioreactor devices and units for the production of biomass and remediation of environmental contamination are provided. The bioreactor devices comprise a membrane photobioreactor (PBR), the PBR comprising a liquid medium, at least one photosynthetic microorganism, and at least one outer membrane layer, wherein the membrane layer is comprised of a material that is permeable to gas transfer across the membrane layer; and further comprise a chamber defining a gaseous atmosphere enclosed within, wherein the PBR is located inside the chamber. The devices also comprise a control system which controls the composition of the atmosphere within the chamber. Gas transfer occurs across the membrane layer of the PBR, between the PBR and the atmosphere comprised within the chamber. Systems comprising the devices are provided as well as methods of using the devices for the production of biomass, remediation of wastewater and removal of atmospheric pollutants.

### FIELD

The present invention relates to photo-bioreactor devices that can be used to generate biomass and assist in environmental remediation. Such devices can also remove gases, such as carbon dioxide and nitrogen oxides, from the environment and can generate oxygen

### BACKGROUND

Due to the global shift away from a reliance on fossil fuel-based energy sources, biomass is becoming increasingly important for energy generation, production of chemicals, production of food and feed ingredients and other industrial and environmental applications. Biomass derived from microorganisms is of particular interest because it can be produced much faster than other types of land-based agricultural biomass, such as corn and soy, and, once harvested, it can be processed (e.g. by fermentation or refinement) to produce biofuels such as biodiesel, ethanol, butanol and methane (biogas) and/or to produce valuable chemicals and nutrients and/or to produce food and feed ingredients.

US2014/186909 describes a photobioreactor capsule made by transparent (or semitransparent) flexible polymer films which is divided into a plurality of adjacent channels, in communication with a fluid distribution structure.

US2015/0230420 refers to a photobioreactor as well as a biogas unit equipped with such a photobioreactor, which uses a transparent pipe system for the flow-through of a culture suspension, configured in the form of levels in order to enable cultivation over several levels.

DE102012013587 relates to a photo-bioreactor comprising a disposable bag defining a reactor chamber bounded by a wall, and light sources arranged in the immediate vicinity of said wall.

US2014/0093924 describes flat panel biofilm photobioreactor systems with photosynthetic, auto fermentative microorganisms that form a biofilm, and which make chemical products through photosynthesis and subsequent auto fermentation.

WO2015/116963 is concerned with bioreactors defining an essentially closed system except for at least one opening that allows for the introduction of gases and/or nutrients. The gas and/or nutrients are introduced in such a way as to provide mixing and aeration of a cell culture in the bioreactor.

US2009/305389 refers to photobioreactors comprising a flexible outer bag, with membrane tubes situated inside the outer bag allowing for introduction of high concentrations of carbon dioxide into the media contained within.

US2012/329147 describes an aquatic algae production apparatus employing a support assembly and a cluster of floating CO₂/O₂ permeable photobioreactors submerged close to the water surface.

US2012/040453 relates to bioreactors comprising at least two chambers separated by an oxygen-permeable membrane using oxygen-carrying molecules to deliver oxygen to a cell culture.

US2015/275161 describes a photobioreactor comprising plastic sheeting coated with a thin layer of a highly dense culture of a photoautotrophic single celled organism.

US2010/261918 is concerned with a process for separating lipid oil from an algal biomass for biofuel production comprising breaking the algae cells and separating lipid oils from the broken cells, with the lipid oils then converted to biofuel.

US2014/144839 refers to apparatus and methods for cultivating microalgae using effluent from sludge treatment, including a microalgae cultivation reactor supplied with the effluent from aerobic digestion chambers.

U.S. Pat. No. 8,409,845 describes flexible bags with CO₂/O₂ exchange membranes, suspended in a first liquid (e.g., seawater) which cultivate algae inside in a second liquid to produce hydrocarbons.

Photobioreactors (PBRs) consume CO₂ and produce O₂ which must be introduced and removed respectively from the liquid media contained within.

High concentrations of CO₂ can encourage the growth of photosynthetic microorganisms, as can an array of other parameters such as optimal temperature, optimal pH, and the present of high levels of nutrients and illuminance. CO₂ is constantly consumed by photosynthetic organisms in the liquid media of a membrane based PBR, and atmospheric CO₂ partial pressure (pp) is not always high enough to maintain sufficient CO₂ transfer through the membrane to replenish or maintain high concentrations of CO₂. As a result, optimal CO₂ concentration may not be maintained within the liquid media. This shows the need to effectively and economically control the CO₂ concentration in the liquid media of the PBR. In view of this problem there has been a tendency in the art to move towards immersion of the PBRs in a liquid which allows for more advantageous control of CO₂ pp across the membrane.

There is also a need to provide novel mechanisms for carbon capture and sequestration (CCS), that is, the prevention of CO₂ release or the removal of CO₂ from an atmosphere, to reduce the effects of CO₂-associated climate change. The aim of such mechanisms is to convert CO₂ to a usable or storable form. Atmospheres may comprise a standard environmental atmosphere or atmospheres that have been modified, such as by introduction of effluent gas.

High concentrations of O₂ can be toxic to photosynthetic organisms such as algae, and can decrease growth of such organisms, thereby decreasing biomass production rate. O₂ is produced as a waste product of microbial photosynthesis and therefore must be removed from liquid media to maintain a suitable O₂ level. The concentration of O₂ in atmospheric O₂-saturated water can be higher than the optimal O₂ concentration levels for the growth of photosynthetic microorganisms. Additionally, the differential between O₂ concentration in the liquid media of a PBR and the pp of O₂ in the surrounding atmosphere may not be sufficient to enable rapid and effective depletion of O₂. Thus, there is also a need to control the concentration of O₂ in the liquid media and/or to remove the excess O₂ in an effective and economical way. Again, the one standard approach in the art for addressing this problem is to ensure that a membrane PBR is surrounded by a liquid.

pH is another factor important for the optimal growth of photosynthetic organisms. The delivery of gases can be used to control pH levels in the liquid media to reach the desired ideal, with CO₂ being able to affect solution pH, and other possibilities including NH₃ (ammonia).

Certain gases also stimulate specific physiological activity in particular microorganisms, with these gases often not present in a natural atmosphere. As a result, the effective and economical delivery or removal of specific gases to or from the liquid media provides a means of stimulating specific microbial activity.

Change in the gas concentrations in liquid media can arise from a wide array of sources, such as environmental or climatic changes, different applications or installations of the PBR, differences in the microorganisms contained within it, the changing of culturing parameters or the biomass produced, or change in microbiological activity.

Thus, there is a need to adaptably control the concentration of certain gases, including but not limited to CO₂ and O₂ within the liquid media comprised within a membrane PBR, in order to (i) stimulate specific microbial activity and/or (ii) increase biomass production rate and/or (iii) change the chemical composition of the biomass produced.

The present invention addresses the problems that exist in the prior art, not least the production of valuable products from biomass, improvements in CCS and more efficient control of PBR systems. These and other uses, features and advantages of the invention should be apparent to those skilled in the art from the teachings provided herein.

### SUMMARY

According to a first aspect of the invention, there is provided a device for the production of biomass, the device comprising a membrane photobioreactor (PBR), the PBR comprising a liquid medium, at least one photosynthetic microorganism, and at least one outer membrane layer, wherein the membrane layer is comprised of a material that is permeable to gas transfer across the membrane layer. The device also comprises a chamber defining a gaseous atmosphere enclosed within, wherein the PBR is located inside the chamber; and also a control system which controls the composition of the atmosphere within the chamber. Gas transfer occurs across the membrane layer of the PBR, between the PBR and the atmosphere comprised within the chamber. Suitably the chamber is substantially gas impermeable.

In some embodiments of the invention the chamber is comprised of a plurality of walls and at least one wall, or a portion thereof, permits the transmission therethrough of visible light into the interior of the chamber. The chamber may further comprise a source of illumination.

In some embodiments of the invention the walls of the chamber may be substantially rigid. The walls of the chamber may comprise ethylene tetrafluoroethylene (ETFE).

In some embodiments of the invention the membrane layer of the PBR may be translucent, typically substantially transparent, and may comprise polysiloxane. The PBR may be substantially surrounded on all sides by the atmosphere within the chamber.

In some arrangements of the invention, multiple PBRs may be located inside the chamber, and the liquid media of the PBRs may be in fluid communication. Other arrangements may comprise multiple devices according to any of the above, wherein the liquid media of multiple PBRs are in fluid communication; and the atmospheres of multiple chambers are in fluid communication.

In some embodiments of the invention the at least one photosynthetic microorganism may be selected from one or more of the group consisting of: Haematococcus sp., Haematococcus pluvialis, Chiorella sp., Chiorella autotraphica, Chiorella vulgaris, Scenedesmus sp., Synechococcus sp., Synechococcus elongatus, Synechocystis sp., Arthrospira sp., Arthrospira platensis, Arthrospira maxima, Spirulina sp., Chiamydomonas sp., Chiamydomonas reinhardtii, Dysmorphococcus sp., Geitlerinema sp., Lyngbya sp., Chroococcidiopsis sp., Calothrix sp., Cyanothece sp., Oscillatoria sp., Gloeothece sp., Microcoleus sp., Microcystis sp., Nostoc sp., Nannochioropsis sp., Anabaena sp., Phaeodactylum sp., Phaeodactylum tricornutum, Dunaliella sp., Dunaliella salina.

In some embodiments, the device of the invention may be divided into two or more sections to provide at least a first chamber section and a second chamber section.

In some embodiments, the control system is configured to introduce a CO₂-rich gas into the chamber or one or more of the chamber sections. The control system may be configured to introduce an O₂-depleted gas into the chamber or one or more of the chamber sections. In some embodiments the control system may be configured to introduce an effluent gas from an industrial source into the chamber or one or more of the chamber sections.

According to another aspect of the invention there is provided a process for the control of a microbial culture within a membrane photobioreactor (PBR), the PBR comprising at least one outer membrane layer wherein at least one gas can pass across the membrane layer, the process comprising the steps of: providing a microbial culture within the PBR, wherein the microbial culture comprises a liquid medium and at least one photosynthetic microorganism, and is capable of producing biomass; locating the PBR within a chamber, wherein the chamber comprises at least a first inlet, and further comprises walls that define and enclose a gaseous atmosphere within the chamber, which walls in some embodiments render the chamber substantially impermeable to gas; controlling the atmosphere within the chamber by controlling the content of a feed gas entering the chamber through the first inlet; and wherein production of biomass by the microbial culture within the PBR is controlled and/or affected by controlling the atmospheric composition of the atmosphere within the chamber.

A device according to yet another aspect of the invention comprises a membrane photobioreactor (PBR), the PBR comprising a liquid medium, at least one photosynthetic microorganism, and at least one outer membrane layer, wherein the membrane layer is comprised of a material that is permeable to gas transfer across the membrane layer; and further comprises a chamber defining a gaseous atmosphere enclosed within, wherein at least a portion of the PBR is located inside the chamber. In some embodiments at least 30%, typically at least 50%, suitably at least 70%, optionally at least 90% of the PBR is located inside the chamber, and typically substantially all of the PBR is located inside the chamber.

A device according to a still further aspect of the invention comprises a membrane photobioreactor (PBR), the PBR comprising a liquid medium, at least one photosynthetic microorganism, and at least one outer membrane layer, wherein the membrane layer is comprised of a material that is permeable to gas transfer across the membrane layer, and a chamber comprising walls, that define a gaseous atmosphere enclosed within, wherein the PBR is located inside the chamber. In some embodiments the chamber comprises at least upper and lower walls. The upper wall may have a rounded convex shape, or may be tilted relative to the horizontal, to permit fluid runoff under gravity from a surface defined thereon.

### DRAWINGS

The invention is further illustrated by reference to the accompanying drawings in which:

FIG. 1 shows a cross-section (Section A of FIG. 13a) of a device according to an embodiment of the invention having a linear photobioreactor with an inlet and an outlet located on opposite sides, disposed within a gas-filled chamber also provided with an inlet and outlet.

FIG. 2 shows a cross-section of a device according to an embodiment of the invention also illustrating the movement of gases from the atmosphere within the chamber to the PBR and vice versa.

FIG. 3 shows a cross-section of a device according to an embodiment of the invention wherein the chamber is separated into two sections.

FIG. 4 shows a cross-section of a device according to an embodiment of the invention also illustrating the movement of gases from atmospheres comprised within each of the two sections of the chamber into the PBR and vice versa.

FIG. 5 shows a cross-section of an arrangement according to an embodiment of the invention with two PBRs directly connected in series, wherein both PBRs are contained within a single chamber.

FIG. 6 shows a cross-section of an arrangement according to an embodiment of the invention with two PBRs directly connected in series, wherein each PBR is contained within a chamber, the interiors of which are also connected to each other.

FIG. 7 shows a cross-section of an arrangement according to an embodiment of the invention with two PBRs connected in series via a conduit.

FIG. 8 shows a cross-section of an arrangement according to an embodiment of the invention with two PBRs directly connected in series, wherein each PBR is contained within a chamber further separated into two sections, and wherein the interiors of each section are connected with the corresponding section of the other chamber.

FIG. 9 shows a cross-section of an arrangement according to an embodiment of the invention with two PBRs connected in series via a conduit.

FIG. 10 shows a cross-section (Section B of FIG. 13a) of a device according to an embodiment of the invention having a PBR contained within a chamber.

FIG. 11 shows a cross-section of a device according to an embodiment of the invention having a PBR contained within a chamber wherein the chamber is separated into two sections.

FIG. 12 shows a cross-section (Section C of FIG. 13b) of a device according to an embodiment of the invention having a PBR contained within a chamber wherein the chamber is separated into two sections.

FIG. 13a shows the planar sections A and B through a representation of a device according to an embodiment of the invention and is included to aid understanding of the other drawings provided herein.

FIG. 13b shows the planar section C through a representation of the device according to an embodiment of the invention wherein the PBR has a central flow control structure creating a bifurcated channel, and is included to aid understanding of the other drawings provided herein.

FIG. 13c shows the planar section D through a representation of the device according to an embodiment of the invention wherein the PBR or a portion thereof has a flow control structure creating a sinuous or tortuous channel for liquid media to flow through, and is included to aid understanding of the other drawings provided herein.

FIGS. 14a, b and c show the planar section A through a representation of the device according to an embodiment of the invention and are included to aid understanding of the drawings provided respectively by FIGS. 5, 6 and 7.

FIG. 15 shows a cross-section of a device according to an embodiment of the invention having a linear photobioreactor enclosed within a chamber, the walls of the chamber being made up of two layers with an intervening space.

FIG. 16 shows a cross-section of a device according to an embodiment of the invention wherein all but the lower of the walls of the chamber are made up of two layers with an intervening space, with the lower wall made up of a single layer, and this wall being positioned against a surface.

FIG. 17 shows a cross-section of a device according to an embodiment of the invention wherein the upper and lower walls of the chamber are made up of two layers with an intervening space, with the side walls being made of a single layer.

FIG. 18 shows a cross-section of a device according to an embodiment of the invention wherein the upper wall of the chamber is made up of two layers, with the side and lower walls made up of a single layer, and the lower wall being positioned against a surface.

FIGS. 19a and b show schematics of an auxiliary system according to embodiments of the invention which facilitate control of a device's generation and harvesting of biomass.

FIG. 20 shows a cross section of a support member and associated clamping plate for use with a device according to embodiments of the invention.

FIG. 21a shows a cross-section of a device according to an embodiment of the invention showing how adjacent support members co-operate to support the PBRs within a chamber, and also to divide the chamber itself into sections having independently controlled atmospheres.

FIGS. 21b and c show cross-sections of devices (section D of FIG. 13c) according to embodiments of the invention where the PBR is supported within the chamber by one or more suspension members.

FIG. 22a shows a perspective view of support members for use with a device according to embodiments of the invention.

FIG. 22b shows a perspective view of a support member for use with a device according to embodiments of the invention wherein the support member comprises a plurality of with apertures to allow gas communication between adjacent chambers.

FIG. 23a shows a cross-section of a device according to an embodiment of the invention comprising a convex curved upper chamber wall, to encourage runoff under gravity of water, snow, sand and other substances that might deposit on an interior or exterior surface.

FIG. 23b shows a cross-section of a device according to an embodiment of the invention comprising an upper chamber wall which is tilted relative to the horizontal to create a pitch, again to encourage gravitational runoff of water and other substances that might deposit on an interior or exterior surface.

### DETAILED DESCRIPTION

All references cited herein are incorporated by reference in their entirety. Unless otherwise defined, all technical and scientific terms used herein have the same meaning as commonly understood by one of ordinary skill in the art to which this invention belongs.

The present inventor has developed a gas permeable photobioreactor (PBR) device suitable for generating biomass, comprised within a chamber. Advantageously, the atmosphere within the chamber can be controlled in order to supply the PBR device with a gaseous feed of specified composition as well as removing effluent gas. Embodiments of the invention permit the specified composition to comprise an atmosphere that is optimised in order to improve or maximise biomass production within the PBR. Alternative embodiments of the invention permit for the specified composition to comprise an atmosphere that controls growth of or modulates biomolecule synthesis by a microorganism comprised within the PBR. These and other embodiments of the invention are described in more detail below.

The embodiments of the invention are optimised to maximise the efficiency and adaptability of the photosynthetic microorganisms contained within it, and hence to maximise the efficiency of generation of biomass as well as any valuable products comprised within the biomass.

Prior to further setting forth the invention, a number of definitions are provided that will assist in the understanding of the invention.

As used herein, the term “comprising” means any of the recited elements are necessarily included and other elements may optionally be included as well. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

As the skilled person will be aware, the term “photosynthesis” refers to a biochemical process that takes place in green plants and other photosynthetic organisms, including photosynthetic microorganisms including algae and cyanobacteria. The process of photosynthesis utilises light to convert carbon dioxide and water to metabolites and oxygen. As used herein, the term “photosynthetic microorganism” refers to any microorganism that is capable of photosynthesis. As used herein, the related terms “phototrophic” and “photosynthesising” are synonymous with to “photosynthetic” and the two terms can be used interchangeably herein.

The skilled person will also be aware that references to the concentration or percentage of CO₂ (carbon dioxide) in liquid refers to the dissolved inorganic carbon (DIC) of the solution, that is, the concentration of dissolved CO₂ as well as related inorganic species H₂CO₃ (carbonic acid), HCO₃⁻ (bicarbonate) and CO₃²⁻ (carbonate) Similarly, references herein to “gas concentration” and the like are intended to include any and all ionic species or chemical compounds which form from gases in a liquid or aqueous context, for example ammonium ions (NH₄⁺) as a result of ammonia gas or sulphuric acid (H₂SO₄) as a result of sulphur oxides.

As used herein, the term “translucent” has its ordinary meaning in the art, and refers to a light-pervious material that allows light to pass through, resulting in the random internal scattering of light rays. The term is synonymous with “semi-transparent”.

As used herein, the term “transparent” has its ordinary meaning in the art, and refers to a material that allows visible light to pass through it, such that objects can be clearly seen on the other side of the material, in other words it can be described as “optically clear”. All membrane and non-membrane materials, chamber walls, additional components, control structures, coatings and other materials described herein can be substantially translucent or substantially transparent.

As used herein, the term ‘effluent gas’ means gas produced as a waste product, byproduct or intended product from a natural or human-instigated process, particularly where such gases are enriched in CO₂ and/or depleted in O₂ compared to normal atmosphere. Such processes include but are not limited to combustion, manufacturing, industrial processes, vehicles such as ships, aeroplanes and road vehicles, fermenters, and waste treatment.

As used herein, the term “permeable” or “gas permeable” means a material that allows gases, in particular oxygen (O₂), carbon dioxide (CO₂), nitrogen (N₂) and, optionally, methane (CH₄) to be transferred from one side of the material to the other, in either or both directions. As used herein, the related terms “breathable” and “semipermeable” are synonymous with “permeable” and the two terms can be used interchangeably herein. Typically, the material is in the form of a sheet, film or membrane. The permeation is directly related to the concentration gradient of the permeant (such as gas), a material's intrinsic permeability, and the diffusivity of the permeant species in the membrane material.

Permeability of a gas through a specific material is measured herein in Barrers. The Barrer measures the rate of a gas flow passing through an area of material with a thickness, driven by a given pressure. Barrer is defined as:

$$1 Barrer = {10}^{- 10} \frac{{cm}_{STP}^{3} \cdot cm}{{cm}^{2} \cdot s \cdot cm Hg}$$

It will be appreciated that the Barrer is the most common measurement of gas permeability in current usage, particularly in relation to gas-permeable membranes, however permeability may also be defined by other units, examples of which include kmol·m·m⁻²·s⁻¹·kPa⁻¹, m³·m·m⁻²·s⁻¹·kPa⁻¹, or kg·m·m⁻²·s⁻¹·kPa⁻¹. ISO 15105-1 specifies two methods for determining the gas transmission rate of single-layer plastic film or sheet and multi-layer structures under a differential pressure. One method uses a pressure sensor, the other a gas chromatograph, to measure the amount of gas which permeates through a test specimen. Other equivalent measurements of gas-permeability are known to the skilled person and would be readily equivalent to Barrer measurements described herein.

As used herein, the term “biomass” refers to any living or dead microorganism, including any part of a microorganism (including metabolites and by-products produced and/or expelled by the microorganism). In the context of the present invention, the term “biomass” includes, in particular, the synthetic products of photosynthesis, as described above.

As used herein, the term a “device” may be comprised of one “unit”, or may comprise an array or combination of a plurality of “units”.

As used herein, the term ‘chamber’ also refers to a ‘gas chamber’ and the two terms can be used interchangeably herein.

As used herein, the term “fluid” refers to a flowable material, typically a liquid and suitably liquid media, which is comprised within the units, and thus the devices of the invention. “Fluid” may also be used to describe a gas, such as the atmosphere which is comprised within the chambers of the invention.

As used herein, the term “liquid media” has its usual meaning in the art and is a liquid used to grow the microorganisms and which contains the microorganisms. The liquid media can comprise one or more of the following: fresh water, salty water, saline, brine, sea water, waste water, sewage, nutrients, phosphates, nitrates, vitamins, minerals, micronutrients, macronutrients, metals, digestate, fertilisers, microorganisms growth medias, BG11 growth media, and microorganisms.

As used herein, the related terms “photo-bioconverter” and “photo-bioreactor” are synonymous and the two terms can be used interchangeably herein.

As used herein, terms relating to the orientation of the device of the invention are generally used in their commonly held meanings, but are also intended to vary as appropriate depending on the particular intention or configuration of the invention. Thus, terms such as upper, top and above may refer to directions away from the Earth's gravity, but in some embodiments may refer to directions towards the primary light source used by the invention, for example if the invention is used as a façade for a building. Similarly, terms such as lower, bottom and below refer to directions towards the Earth's gravity and/or away from a primary light source.

The membrane based photobioreactors (PBRs) of the type described and utilised herein may be substantially as described in the present applicant's co-pending International (PCT) patent application no. PCT/GB2016/053786.

The transfer of carbon dioxide gas into PBRs is usually achieved through the use of aeration technologies, such as by compressing CO₂ or air and delivering the compressed gas into the liquid media through nozzles, or by bubbling or sparging the gas into the liquid media (see for example US2015/0230420, WO2015/116963). These techniques, using CO₂-containing or other gas mixtures, can also work to remove excess O₂ (see for example US2015/0093924).

Techniques of this kind can be disadvantageously inefficient in both energy requirements and infrastructure cost. It is estimated that in some PBRs only a small proportion of CO₂ that is bubbled through a liquid becomes successfully dissolved; consequently the remaining CO₂ is wasted, leading to a waste of energy and inefficient CO₂ uptake. Likewise O₂ removal by this technique is limited by the O₂ which can be trapped in the bubbles produced, which provide only a limited surface area for effective gas exchange.

A benefit of the present invention relates to the high energy costs, operational costs and capital costs for controlling gas concentrations associated with aeration and compression devices of CO₂ (or air mixtures) in standard PBRs as described previously. The present invention enables, in part, much more efficient gas-transfer control in the liquid media, including on a large scale, and provides greater versatility compared to systems that require devices for controlling aeration and compression of feed gases administered directly to the liquid media. The operational complexity and extra weight associated with compression and aeration techniques is also avoided. Gas which has been pressurised to a lower pressure than would be necessary in using other PBR technologies may also be used without the need for further pressure. Due to the nature of the invention, the natural expansion properties of gas mean that supplied gas can be easily supplied and expand to rapidly change the composition of the entire chamber. This provides a further benefit, as the gas concentration within the chamber can be relatively easily controlled on a large scale, and by extension the gas concentration in the liquid media can be controlled on the same scale.

Another benefit of the present invention is in increasing the robustness and environmental resistance of a PBR comprised within an assembly. The walls of the chamber may be configured to provide thermal insulation against external factors such as changing environmental or seasonal conditions. This insulation also decreases the energy necessary for the maintenance of the temperature of liquid media comprised with the PBRs. Physical protection of the potentially fragile membrane of the PBR is also provided against factors such as weather, wind or hail, or animal damage. The provision of an additional barrier also acts to contain spills from the PBR into the environment.

Thermal insulation may also be provided by this invention beyond the device itself. It is envisioned that some embodiments of the invention may be configured for installation on the roofs or facades of buildings, thereby providing an added benefit of insulation to the buildings on which they are installed. For this purpose the surface of the chamber in contact with the building can be replaced with or additionally comprise an insulating material such as cork, bitumen, glass fibre, or any other highly insulating material and/or coatings and/or composites for constructions.

According to one embodiment of the invention, a device is provided that comprises a membrane PBR enclosed inside a chamber. The chamber comprises inner surface walls that cooperate to define the chamber in which a gaseous atmosphere is contained. The (membrane) PBR is entirely enclosed within the chamber. The PBR can be located in contact with an inner surface wall, such as the bottom surface of the chamber. Alternatively, the PBR can be suspended or otherwise positioned substantially centrally within the chamber such that the majority of the outer surface of the PBR membranes are in contact with the atmosphere contained within the chamber, or can rest on fins or protrusions attached to the lower internal wall and/or any other internal wall of the chamber to allow gas to circulate around and across the outer surface of the PBR, or can rest on a net, or a series of cords, strings or cables attached to the side internal walls of the chamber, and/or on any other internal wall of the chamber.

In a further embodiment of the invention, the PBR is partially enclosed within the chamber such that only a portion of the PBR is comprised, and a portion is exposed to the general atmosphere. Suitably, in some embodiments at least 50%, suitably at least 70%, and optionally at least 90% of the PBR is located inside the chamber. In particular embodiments, substantially all of the PBR is located inside the chamber.

The chamber is filled with a gas mixture comprising CO₂ in higher concentration to that of the liquid media, increasing the concentration differential between the liquid media and the surrounding atmosphere. In this way the gas-transfer rate of CO₂ through the membrane into the liquid media is increased.

As the CO₂ (in all its possible forms that can be taken up by photosynthesising microorganisms) in the liquid media is consumed by the photosynthetic microorganisms comprised within, and more CO₂ passes across the membrane of the PBR from the atmosphere within the chamber to the liquid media, the CO₂ gas transfer rate will decrease over time as the concentration differential stabilises to an equilibrium state. To overcome the tendency toward equilibrium, the gas mixture comprising CO₂ can be continuously or intermittently delivered through a gas chamber inlet, and a similar volume of gas can be removed through an outlet, typically using a controlled valve such as a solenoid valve and/or a pressure sensitive valve. Optionally the valve can be closed when the gas mixture is delivered, to pressurise the gas chamber above ambient standard atmospheric pressure and so further increase gas transfer rate across the gas-permeable membrane of the PBR.

The gas mixture introduced into the gas chamber may also comprise a lower concentration of O₂ than that found in the liquid media and/or than atmospheric O₂ levels, in order to increase the O₂ depletion rate from the liquid media. Alternatively, O₂ can be removed from the liquid media by the introduction into the gas chamber of inert gases such as nitrogen, helium, argon or methane and/or CO₂ in order to increase the O₂ concentration differential between the atmosphere and the liquid media.

In some embodiments, the gas chamber may be separated into two or more sections, referred to herein as first and second chambers etc., into which different gases or gas mixtures can be introduced. For example, the first chamber can contain a CO₂-enriched gas mixture, while the second may contain an O₂-depleted gas mixture such as N₂-rich gas for the effective removal of O₂. In certain embodiments of the invention the PBR provides an intervening barrier between the first and second chambers (and further chambers if required). Hence, in this embodiment of the invention the first and second chambers are defined by exterior walls of the chamber in combination with the membrane wall of the intervening PBR.

The gas can be moved inside the chamber passively by gas expansion, or by using a low energy method which reduces CO₂ feed delivery costs such as a fan, turbine or other impeller. Alternatively the gas can be compressed prior to introduction into the gas chamber.

The internal environment of the chamber can be controlled internally or by controlling the gas supply and/or the gas discharge. For example, the humidity of the atmosphere within the chamber can be controlled by the presence of a desiccating agent installed in the gas inlet, or by a desiccating agent or material or coating placed inside the chamber itself or within an attached auxiliary system. For example the chamber air can be circulated to a dessicant for drying, before being returned to the chamber; typically the desiccant can be in the form of a honeycomb wheel.

At least a portion of the walls that define the chamber material is transparent or translucent, to allow the effective transmission of light such that the PBR comprised within the chamber may function. In some embodiments, at least a portion of one or more of the walls, for example the wall located furthest from the light source, is reflective, in order to increase the passage of light through the PBR. In some embodiments, at least about 10%, at least about 20%, at least about 30%, at least about 40%, at least about 50%, at least about 60%, at least about 70%, at least about 80%, at least about 90% or at least about 100% of the area of the walls may be permeable to light.

‘Switchable glass’, ‘Smart glass’ or similar materials may be used in the invention. These are materials (which can be but are not limited to being rigid like glass, flexible like a polymer film or a coating) whose light transmission properties are altered when voltage, light or heat is applied. These may be of particular use in areas with high light exposure, for example to reduce damage to the materials or the microorganisms as a result of especially high light. Typically, the material changes from substantially translucent, and/or with a reflective optical property (similar to a mirror finish) to substantially transparent, changing from blocking some (or all) wavelengths of light to letting light pass through. Examples of technologies that may be used in pursuit of the above include but are not limited to electrochromic, photochromic, thermochromic, suspended particle, micro-blind and polymer dispersed liquid crystal devices.

Suitably, the walls of the chamber are substantially gas-impermeable and the chamber as a whole is substantially air-tight, to prevent loss or contamination of the controlled atmosphere within.

The walls of the chamber can be composed or defined by the structures or body assemblies of vehicles, industrial machines, ships, spaceships or spacecraft, submersible vehicles, wall cavities, containers, underground chambers, architectural structures, building rooms and/or switch houses.

In these and/or other cases, the chamber walls could comprise materials which are not transparent/translucent. In such cases auxiliary light sources inside the chamber may be used. These auxiliary light sources could be LEDs/OLEDs or fluorescent tubes, or could be natural light channelled by fibre optics and/or optic assemblies. Similarly in cases where the chamber walls are translucent/transparent but the device is located inside or is otherwise remote from natural light, such auxiliary light sources may be used.

The translucent/transparent portion which permits transmission of light into the chamber can be composed of any suitable translucent/transparent material. The chambers can be comprised entirely of the translucent/transparent material, or can be supported on a support structure such as a scaffold or frame, as discussed below. Suitably the material is substantially gas-impermeable, strong, light, and possesses good thermal insulation properties. Optionally the material is provided in sheets and/or films. In some embodiments the material is non-flexible, non-elastic, transparent and strong, for example comprising glass, high performance glass, low iron glass with very high solar energy transmittance (Pilkington Sunplus™), glass composites, reinforced glass composites with increased strength, impact proof glass composites, low reflectance glass, high light transmittance glass, double glazing style glass and/or triple glazing with or without vacuum/argon/air in between, or glass composites made of several layers of different materials to increase strength and/or light transmittance, or electrically switchable smart glass.

In other embodiments the chamber wall material is flexible and elastic, for example comprising ethylene tetrafluoroethylene (ETFE), acrylic/PMMA, polycarbonate and/or other plastics, plastic composites.

The suitable properties of ETFE include its translucency and/or transparency, very high light transmittance, and ultraviolet resistance. ETFE is also advantageously recyclable, easily cleanable (due to its non-adhesive surface), elastic, strong and light, with good thermal insulation, high corrosion resistance and strength over a wide temperature range. Employing heat welding, tears can be repaired with a patch or multiple sheets assembled into larger panels.

Acrylic is suitable as chamber wall material due to its strength, high transparency, and resistance to weathering and ultraviolet radiation.

In specific embodiments of the invention use of flexible and/or elastic material allows for the chamber to be inflated by supplying an atmosphere within the chamber that has a relative positive pressure compared to the surrounding atmosphere outside of the device. Alternatively, gas expansion within the chamber due to an increase in temperature may also cause a corresponding increase in relative positive pressure. In specific embodiments of the invention the use of flexible and/or elastic materials will allow to create a convex, domed, cambered, or otherwise protuberant shape to the upper wall of the chamber (relative to a position outside the chamber) either as a result of positive pressure inside the chamber relative to the surrounding atmosphere (that is, inflation of the chamber by the gas supplied) or by using auxiliary structures attached to the walls of the chamber, to create the convex shape. This can be helpful to avoid the formation of “puddles” of rain, snow, leaves, powder, sand or other detritus which could cause a barrier to light reaching the PBR. Moreover the convex shape will facilitate the self-cleaning of the material when raining and/or facilitate manual/automatic cleaning performed by the plant operators or automatic cleaning system. For similar reasons, in other embodiments of the invention any upper surfaces of the chamber may be tilted slightly relative to the horizontal, for example by having side walls of the chamber of different heights.

Another advantage of such an arrangement is to enable a measure of control over internal chamber humidity—moisture in the chamber atmosphere may condense on the inside of chamber walls, especially if the inside of the chamber is warmer than the outside atmosphere. With convex or tilted upper walls any condensation can be encouraged to run away from the upper walls of the chamber, reducing the interference on light transmission that might occur.

The transparent/translucent material can be coated or treated to affect its optical or chemical properties. For example, the material can be coated to decrease light reflectance, with materials with good transparency/translucency, and/or with gas-impermeable materials. Coatings may confer voltage, light or heat-dependent properties on the material, such as set forth above.

Coatings, chemical modifications or films applied to the material can be used to convert electromagnetic radiation from the visible or invisible wavelengths outside the photosynthetic spectrum into frequencies suitable for photosynthesis or any intended wavelength for example by using optical materials comprising engineered nanodots and/or engineered quantum dots and/or micro and nano optics and/or molecules that change optical properties when charges are applied to and/or removed from the molecule, such as by applying a voltage. Coloured coatings, chemical modifications or coloured films applied to the material can be used to shield specific wavelengths to enable to other wavelengths to reach the liquid media, this technique can be used to promote specific biological activity therefore to increase the production of specific products in the biomass for example by using optical colour filters films and/or optical materials comprising engineered nanodots and/or engineered quantum dots and/or micro and nano optics and/or molecules that change color when charges are applied to and/or removed from the molecule possibly by applying a voltage. For example a red coloured film can be applied on the transparent/translucent material to let substantially only red light reach the liquid media, therefore promoting the production by the photosynthesising microorganisms of pigments that absorb mostly red light, for example the pigment phycocyanin.

Graphene coatings may be used due to its transparency to reinforce the material, to provide antimicrobial growth coatings, to provide electrical conductance that can then help detect breakages (e.g. tearing) of the material. Coatings, treatments, paints or films to reduce mould, bacteria and fungi growth can also be applied to the inside surface of the chamber. Specific materials intended to prevent mould or any microbial growth can be used as components of the chamber. The transparent/translucent material can also comprise graphene, carbon nanotubes and/or graphite for reinforcement, or to enable a thinner and lighter wall material to be used.

It is envisaged that the inside of the chamber may be easily accessed for maintenance purposes by removal of one or more of the walls that comprise the chamber.

According to one embodiment of the invention, the PBR of the device is provided that comprises at least one outer layer that is a membrane layer. The membrane layer or layers may be flexible. At least a part of one of the membrane layers, and optionally substantially all of each of the membrane layers, is permeable to transmission of gases across the membrane. The permeability coefficient of oxygen through the membrane may be not less than about 100 Barrer, typically about 300 Barrer, and suitably about 400 Barrer. In a specific embodiment of the invention the permeability coefficient of oxygen through the membrane is not less than about 500 Barrer and possibly higher. The permeability coefficient of carbon dioxide through the membrane is not less than about 400 Barrer, suitably not less than about 600 Barrer, about 800 Barrer, about 1000 Barrer, 1500 Barrer, about 2000 Barrer, about 2500 Barrer, and typically not less than about 3000 Barrer. In a specific embodiment of the invention the permeability coefficient of carbon dioxide through the membrane is not less than about 3200 Barrer. As used in this context, the phrase “at least a part” means an area of the layer that is of a sufficient size to allow a gas to pass through the outer layer of the PBR. The gas is typically oxygen and carbon dioxide, but not limited thereto, and may comprise nitrogen, nitrogen oxides, sulphur oxides and/or methane.

The PBR may be illuminated from a single direction or from multiple directions. If the PBR is positioned such that it receives light primarily from a single direction and one (first) membrane layer is less transparent or less translucent than another (second) membrane layer, the first membrane layer can be on the side of the PBR which faces the primary light source. In a particular embodiment, the first membrane layer is located on the side of the PBR facing away from the light source.

Typically, the membrane layer is at least translucent, and is suitably substantially transparent.

Typically, a membrane layer comprises one or more gas permeable materials. It is important that the gas permeable material is not permeable to liquids, to prevent liquid media inside the PBR leaking to the outside. The gas permeable material can be porous (including microporous structure gas permeable materials) or non-porous. Gas permeable materials are referred to as porous if the gas particles can migrate through direct movement through a microporous structure. If the gas permeable material is porous, it is important that it is substantially impermeable to liquids. Suitably, the gas permeable material is non-porous, this to avoid also liquid permeation through the gas permeable material and to avoid lower transparencies which could relate to the porosity of the material.

The gas permeable material may be a polymer, such as a chemically-optimised gas permeable polymer. Chemically-optimised polymers may be advantageous over corresponding unmodified polymers because they may be cheaper, more resistant to tear, hydrophobic, antistatic, more transparent, easier to fabricate with, less brittle, more elastic, more permeable to gases and selectively permeable to specific gasses. Chemical modifications on polymers may be performed in any way a skilled person will know such as by modifying the chemical composition of the monomer, the back bone chain, side chains, end groups, and/or the use of different curing agents, crosslinkers, fillers, processes of vulcanisation, manufacture, fabrication, and other methods.

The membrane layer can comprise any suitable gas permeable material including, but not limited to: silicones, polysiloxanes, polydimethylsiloxanes (PDMS), fluorosilicone, organosilicones, cellulose (including plant cellulose and bacterial cellulose), cellulose acetate (celluloid), nitrocellulose, and cellulose esters.

In a suitable embodiment, the membrane layer comprises polysiloxanes, optionally optimised polysiloxanes. The polysiloxanes may be chemically-modified or machine-modified. Typically, the membrane layer comprises polysiloxane elastomers. It has been found that polysiloxanes are good candidates for gas permeable membranes thanks to the Si—O bonds into the polymer structure which facilitates higher bond rotation, increasing chain mobility, and thereby increasing levels of permeability. Polysiloxane elastomers (such as silicone rubber) are also flexible, tolerant to UV radiation and resilient materials.

In an embodiment, the membrane layer comprises polydimethylsiloxanes (PDMS), suitably optimised polydimethylsiloxanes. Typically the membrane layer comprises polydimethylsiloxane (PDMS) elastomers. Polydimethylsiloxanes (PDMS) can take form of an elastomer, a resin, or a fluid. The PDMS elastomer is formed using a cross-linking agent. PDMS is a typical gas permeable material because of its very high oxygen and carbon dioxide permeability, its optical transparency and its tolerance to UV radiation. These elastomers typically do not support microbiological growth on their surface, and so avoid uncontrolled biofilm growth and/or biofouling which can reduce the efficacy of the device to generate biomass (shielding light). Optionally a biofilm growth can be facilitated by utilising biological supports and/or additional components as described below. Additionally, polydimethylsiloxanes (PDMS) elastomers are flexible and resilient materials.

The polydimethylsiloxanes (PDMS) may be chemically-modified or machine-modified to increase its gas permeability and/or to change its properties. PDMS elastomers typically have an oxygen permeability of at least 350, at least 400, at least 450, at least 550, at least 650, at least 750, suitably at least 820 Barrers and a carbon dioxide permeability of at least 2000, at least 2500, at least 2600, at least 2700, at least 2800, at least 2900, at least 3000, at least 3100, at least 3200, at least 3300, at least 3400, at least 3500, at least 3600, at least 3700, at least 3800, suitably at least 3820 Barrers. The properties of the PDMS used in embodiments of this invention can be optimised through chemical, mechanical and process-driven interventions related to but not limited to the molar mass (Mₘ) of polymer chains, the dispersity in the polymer (dispersity is the ratio of the weight average molar mass to number average molar mass), the temperature and duration of the heat treatment during curing, the ratio of the cross-linking agent to PDMS, the cross-linking agent chemical composition, different end groups (such us methyl-, hydroxy- and vinyl-terminated PDMS) which can influence the way in which end-linked PDMS structures form during cross-linking.

The membrane layer may be no more than about 1000 μm in thickness, suitably no more than about 800 μm, about 600 μm, about 400 μm, about 200 μm and typically no more than about 100 μm, optionally no more than about 50 μm, suitably no more than 25 μm or less.

In another embodiment, the membrane layer comprises bacterial cellulose. While bacterial cellulose has the same molecular formula as plant cellulose, it has significantly different macromolecular properties and characteristics. In general, bacterial cellulose is more chemically pure, containing no hemicellulose or lignin. Furthermore, bacterial cellulose can be produced on a variety of substrates and can be grown to virtually any shape, due to the high moldability during formation. Additionally, bacterial cellulose has a more crystalline structure compared to plant cellulose and forms characteristic thin ribbon-like microfibrils, which are significantly smaller than those in plant cellulose, making bacterial cellulose much more porous. The skilled person will be aware of a number of bacterial systems that are engineered to optimise cellulose production, such as the cellulose biosynthetic system of Acetobacter sp., Azotbacter sp., Rhizobium sp., Pseudomonas sp., Salmonella sp., and Alcaligenes sp., which can be expressed in E. coli, for example. Bacterial cellulose can be treated such that its surface provides a chemical interface to enable bonding with molecules.

Other layers of the PBR may also be a membrane layer—i.e. gas permeable layer—as defined above, or they may be comprised of a non-membrane layer, comprising any suitable material, such as a natural or synthetic material. Suitably, the layers are at least translucent, and are typically transparent. The layers are suitably breathable.

In a typical embodiment, all layers of the PBR are gas permeable membrane layers as defined herein. In other embodiments, the membrane PBR comprises a single layer, such as a tube or a single membrane formed of a continuous layer or a single layer folded on and sealed to itself in one or more places to create the PBR.

The microorganism contained within the PBR of the device is typically capable of performing photosynthesis or other reactions that are dependent upon the presence of an electromagnetic energy source. Any microorganism that is capable of photosynthesis is referred to herein as a photosynthetic microorganism. In a suitable embodiment, the photosynthetic microorganism is selected from micro-algae (such as green, blue-green, golden and red algae), phytoplankton, dinoflagellates, diatoms, bacteria and cyanobacteria, such as Spirulina sp. The microorganism may be a wild-type or genetically-modified strain. A single device according to embodiments of the invention may comprise one or more different types of microorganisms.

Typically, at least one microorganism is a Haematococcus sp., Haematococcus pluvialis, Chiorella sp., Chiorella autotraphica, Chiorella vulgaris, Scenedesmus sp., Synechococcus sp., Synechococcus elongatus, Synechocystis sp., Arthrospira sp., Arthrospira platensis, Arthrospira maxima, Spirulina sp., Chiamydomonas sp., Chiamydomonas reinhardtii, Dysmorphococcus sp., Geitlerinema sp., Lyngbya sp., Chroococcidiopsis sp., Calothrix sp., Cyanothece sp., Oscillatoria sp., Gloeothece sp., Microcoleus sp., Microcystis sp., Nostoc sp., Nannochioropsis sp., Anabaena sp., Phaeodactylum sp., Phaeodactylum tricornutum.

Dunaliella salina, some Arthrospira platensis, some Nannochioropsis sp. and Synechococcus marinus are typical microorganisms in embodiments where the liquid media passing through the channels in the device comprises sea water, salt water or brine.

Some photosynthetic organisms, whether native strains or genetically modified or engineered strains, can have the ability to uptake air-pollutants such as NO₂ (and other NOx such as NO, N₂O₂, N₂O₃, N₂O₅), SO₂ (and other SOx such as S₂O₂, SO, SO₃), VOCs, NH₃, or ‘greenhouse’ gases other than CO₂ such as N₂O. If so, these gases can be pumped in the gas chamber to then be transferred in the liquid media. These gases can also come from effluent gases.

In some embodiments, the photosynthetic microorganisms of the PBR are genetically modified to possess a specific trigger that is activated by exposure to a gaseous or vaporized stimulant that can be delivered into the atmosphere comprised within the chamber. When this stimulant is introduced into the chamber it diffuses across the membrane of the PBR and is delivered into the liquid media. The stimulant acts as a trigger and induces the photosynthetic microorganisms to react in a predetermined manner as intended by the genetic intervention. For example, the stimulant may induce the production or cease of production of a particular metabolite and/or may change the production rates of particular metabolites.

The above descriptions regarding the provision of CO₂-enriched and/or O₂ depleted atmosphere within the chamber is applicable to all other suitable gases, the control of which can be used for a variety of purposes.

Gases can be introduced into the chamber to control the pH of the liquid media comprised within the PBR. According to specific embodiments of the invention the concentration of CO₂ and ammonia (NH₃) within the atmosphere may be used to control the pH of the liquid media.

As described above, microorganisms may be modified to respond to the presence or absence of certain gases by changing their physiological processes, and the gas mixture supplied to the atmosphere comprised within the chamber can be controlled to provide or remove such a gas.

The composition and/or quantity of the gas mixture supplied to the device may be controlled and moderated in response to a change in one or more parameters measured within the liquid media within the PBR, and/or in response to the metabolic or other physiological state of the photosynthetic microorganisms comprised within the PBR. For example, parameter changes including a pH change in the liquid media could lead to the provision of a pH-affecting gas. Alternatively, the detection of a low CO₂ concentration in the liquid media could lead to the supply of an increased level of CO₂ in the CO₂ enriched gas. Monitoring of the status of the liquid media and/or photosynthetic microorganisms may be carried out through an auxiliary system controlling the device (see below).

A CO₂ rich atmosphere can be provided within the chamber by introducing effluent gases obtained from industrial sources, for example, from boilers, power generators, combined heat and power generators (CHP units), industrial processes, fermentation tanks including breweries, wastewater treatment processes/activated sludge/denitrification, or anaerobic digesters, or any type of vehicles or combustion engines. Effluent gas may need to be pre-treated before its delivery to the gas-chamber, for example to remove substances which may be toxic to the photosynthetic microorganisms or that may affect the cleanliness or transparency of the PBR or chamber surfaces. Pre-treatment of gaseous feed to the chamber may include any suitable technologies or strategies such as high efficiency particulate air (HEPA) filters and/or activated carbon filters, and can work to remove specific air pollutants, volatile organic compounds (VOCs), particulate matter of various grades (for example PM1, PM2,5, PM10), soot, and any other undesirable or otherwise toxic content.

According to a specific embodiment of the invention, a feed gas can be delivered in the chamber in the opposite direction of the overall direction of liquid media flow in the PBR. In this way a counterflow arrangement can be established wherein the feed gas with the highest CO₂ concentration can be brought into contact with the liquid media with the lowest dissolved CO₂ concentration (due to photosynthesis occurring during liquid media flow through the PBR system), and likewise the gas with the lowest O₂ concentration contacts the liquid media with the highest dissolved O₂ concentration. This increases the concentration differential of the gases and so improves gas transfer efficiency.

The device can comprise a support structure that includes a frame, scaffold and/or manifold which serves to elevate and/or support the PBR within the chamber—as well as supporting a plurality of PBRs within a plurality of chambers where an array is comprised within the device. The support structure maintains the shape and structure of the chamber itself, and/or in terms of directing flow of the gaseous atmosphere around the PBR comprised within the chamber. Additionally, the support structure may further aid in the attachment of the device to a mount or other surface, and in providing stability of the device as a whole.

In a specific embodiment of the invention a support structure can be comprised of an extrusion of a rigid solid material, and preferably lightweight, as described in the exemplary device below. The support structure has no need to be transparent, although it can be, and may be manufactured from any suitable material, which is typically a strong, light and non-toxic material, with high resistance to oxidation, corrosion, extremes of temperature and ultraviolet radiation. The support structure can comprise a substantially solid material, or can comprise a porous structure to decrease its weight while maintaining strength.

Suitably, the support structure can comprise plastics, such as bioplastics, thermoplastics, thermosetting polymers, amorphous plastics, crystalline plastics, synthetic polymers such as acrylics, polycarbonates, polyesters, polyurethanes carbon fibre composites, Kevlar composites, carbon fibre and Kevlar composites or fibre glass; metals or metal alloys such as steel, mild steel, stainless steel, aluminium or titanium; natural materials such as wood or coated wood; or carbon-based materials such as graphene, carbon nanotubes or graphite.

The PBRs of the device may be connected to an auxiliary system which controls the supply and condition of the gas and/or liquid media used. Depending on the application of the device, the auxiliary system can be of any degree of complexity and composed by any kind of auxiliary components.

In a suitable embodiment of this invention, the device is connected to an auxiliary system mainly composed by conduits for gas and for liquid media, water tanks, gas tanks or canisters, pumps for gas and liquid media, valves, biomass-separators, artificial lighting systems (especially if natural light is not present), water temperature control systems, sensors and computers.

The conduits and reservoirs (water tanks) can be of any type and of any suitable material.

The pumps can also be of any type; typically the liquid pumps are peristaltic pumps which can reduce the contamination risk of the liquid media and the breakage of the cells of the microorganisms used due to the use of a peristaltic tube which is the only component in contact with the liquid media. In some embodiments diaphragm pumps (also known as membrane pumps) can be used. Diaphragm pumps create relatively little friction with the liquid media and so can have advantages in the reduction of cell breakage and the risk of contamination.

Biomass-separators can be of any type known to the skilled person; suitably the biomass-separator is a centrifuge type bio-separator, a filtering system comprising small-aperture meshes, and/or microfiltration/nanofiltration devices, and/or a sedimentation device, and/or clarification process. Multiple biomass-separation devices can be installed in series, for example an initial clarification process or microfiltration device followed by a centrifuge.

The water temperature control can be of any type known to the skilled person; typically it comprises a heating component which is suitably installed around parts of the conduits and/or on the water tank. The heating components can be of any type, and suitably can comprise heat-exchange mechanisms. In particular, it is envisioned that heat exchange may be used to maintain optimum liquid media temperature for the photosynthetic microorganisms. Excess heat from the liquid media generated by physiological processes or high environmental temperatures may be used to heat water for domestic or industrial purposes, or water from sources such as drain water, storm water, sewage water and/or grey water may be used to remove excess heat. Likewise, liquid media may be heated when necessary using heat generated from domestic or industrial sources. Heat exchange devices can be of any suitable type, such as double pipe heat exchangers for low volumes, or plate heat exchangers for larger volumes, due to their size and economy. Heat exchange is suitably carried out in the location of the auxiliary system, before the liquid media arrives in the PBRs.

An artificial lighting system can be used that comprises any artificial light source types known to the skilled person, suitably the lighting system is comprises LEDs, typically the artificial light source is designed and/or controlled to emit specific wavelengths of electromagnetic radiation (Light) corresponding to the photosynthetically active radiation (PAR) needs of any photosynthetic microorganisms contained within the device and/or to promote specific biological activity, thereby increasing the production of specific products in the biomass, for example by using LEDs that emit specific wavelengths. For example an LED-based light source can emit wavelengths between approximately 620 nm and 750 nm (red light) to promote the production in the microorganisms of pigments that absorb mostly red light, such as the pigment phycocyanin. Artificial lighting systems may be comprised within the support structure that comprise arrays or strips of LEDs or optic fibres. The intensity and quality of the light emitted by the lighting systems could be controlled automatically (following inputs from any kind of sensors like PAR sensors, humidity sensors, temperature sensors, chemical sensors, pH sensors and so on) to promote specific microbial physiological activities and/or to respond to environmental changes and/or to increase or modify the biomass production. Similarly the amount of light transmission (either being natural or artificial light) through a ‘switchable’ or ‘smart glass’ material as discussed above can be automatically controlled for similar reasons.

According to one specific embodiment of the invention, when the biomass concentration in the liquid media comprised within the PBR reaches the desired level, a 3-way valve directs the flow into a biomass-separator which separates at least a part of the biomass from the liquid media, the isolated biomass proceeds into a receptacle for additional processing, while the liquid media is directed back into the reservoir. This action of directing the flow into the biomass-separator can be performed periodically and for a predetermined period of time before the valve changes the flow path into the reservoir again. This timing can be optimised with respect to each application, the microorganism used, the surrounding environment and physical location of the device. In another embodiment instead of a binary switch, the valve can change the aperture of the channel thereby controlling the flow rate and amount of liquid media that is delivered to the biomass separation process.

Nutrients can be periodically introduced in the system directly into the reservoir. Water and/or microorganisms in liquid media, or cleaning fluid, can be similarly introduced.

All sorts of other system components can be utilised, as example a controllable pressure valve or pressure regulator can be placed in the system, in this example the pressure valve can control the volumetric change of the unit through the effects of changes in the liquid or gas pressure. Some valves can control the flow rate into the units.

Supplementary air and/or air enriched with CO₂ and/or other gases can optionally be introduced in the main PBR supply conduit if required. Air vents can be installed in the conduits to remove air that can accidentally enters the hydraulic system, for example during installation of the system, and are typically located in the highest location of the system to facilitate the expulsion of undesirable air.

A cleaning procedure can be actuated to clean and/or sterilise PBR units and/or the conduits and/or the water tank and/or all the auxiliary system and/or the chamber. A “cleaning fluid” can be made of any compound known to the skilled person. It may comprise hydrogen peroxide, ethanol, water, saltwater, detergents, bleach, surfactants, alkali or any other suitable cleaning composition. The cleaning fluid can enter the system through specific conduits (inlets) in any point of the system and can exit at any point of the system (outlets) to permit cleaning in specific locations only, if desired, instead of cleaning the entire system. The cleaning fluid may also be gaseous in nature and can comprise steam, heated air or water vapour, suitably supplied at temperatures above 120° C.

Sensors comprising transparent/translucent electrically conductive materials and/or any other electrically conductive materials can be provided on the transparent/translucent portions or on any other surface of the chamber (inside or outside the chamber) to monitor conditions such as irradiance levels, temperature, humidity or other environmental conditions. These sensors or similar sensors, if located inside the chambers may be used to detect gas concentration levels, humidity and/or temperature in the chamber.

Embodiments and/or the auxiliary system of the invention can include embedded sensors which can be used, for example, to monitor chemical concentrations such as CO₂ concentrations and/or O₂ concentrations in liquid media and/or atmosphere; and/or to monitor temperature and other environmental and biological parameters, such as toxicity levels and/or to monitor the biomass concentration and/or the total cell density and/or the viable cell density and/or the photosynthetic activity of the microorganisms in the liquid media.

Sensors can be embedded entirely or partially in the PBR or the chamber, in the tanks or conduits auxiliary system, and/or in control or support structures and/or be attached to the inside or outside of external layers or on surface of internal additional components.

Sensors can permit the monitoring of the environment inside the PBR of the device, in order to enable control of parameters including, but not limited to, liquid media flow rate, liquid media quality, nutrient levels, temperature, biomass extraction rate, gas mixture, gas flow rate, gas chamber pressure, and lighting intensity (and/or optical shielding such as provided by ‘smart glass’). The purpose of this control is to optimise the photosynthetic efficiency of the photosynthetic microorganisms contained within the device, and/or to stimulate specific metabolic/microbial activities and hence to optimise the efficiency of generation of biomass and/or modify its composition.

Similarly, sensors can permit the monitoring of the environment inside the chamber of the device, in order to enable control of parameters including, but not limited to, gas flow rate, quality, composition, temperature, optical clarity and humidity.

An advantage of some embodiments of the invention is that biomass can be generated continuously within the unit and can be harvested on a continuous basis.

Biomass accumulates in the liquid media within the unit, in some cases in regions of biofilm that form on the surface of components of the device, including the inner surfaces of the two outer layers of the PBR. The biomass can be harvested directly from the liquid media, and optionally also with chemical treatment to facilitate biomass detachment from the inside of the device. Biomass is mostly formed in the system during travel of the liquid media through the PBR, as this is where it is exposed to light and CO₂. In order to purge the device and release biomass, liquid media enters the device via the one or more inlets, passes through the one or more channels and exits the device, together with biomass that is carried in the flow, via the one or more outlets. The outlet can be connected to a suitable receptacle for receiving the harvested biomass.

In some embodiments a biofilm is intentionally grown within the device. In such embodiments the biofilm functions to provide a fixed active photosynthetic microbial surface, which prevents some of the microorganisms from being washed away when the device is flushed through. This facilitates rapid generation of biomass and allows for continuous harvesting of biomass generated in the device. This enables the device to regenerate/replenish biomass quickly, because the microorganisms that remain within the device can continuously generate biomass via photosynthesis (provided that the light conditions allow photosynthesis). Furthermore, new/additional microorganisms do not have to be introduced into the PBR after biomass has been harvested in order for more biomass to be generated.

Alternatively, biomass can be harvested intermittently, on a batch basis. For example, biomass can be harvested from the device of the invention frequently, on an hourly, daily or weekly basis.

The device of this invention can be utilised for many applications. The applications can be of any kind including biomass production, carbon dioxide sequestration, oxygen production, the sequestration of nitrogen oxides or other gases, or where the removal of pollutants is needed, or where waste water treatment is needed, or even for aesthetic or decorative applications such as urban furniture or functional artistic installations. Effluent gases for use in the invention can be supplied from any of these applications, or other local or distant sources; the device can thereby be used as a decarbonising system at locations such as warehouses, breweries, industrial buildings and the like. Similarly, the device can be used in conjunction with transportation vehicles, such as ships, aeroplanes, cars, trucks and other road vehicles. The device can be used indoors and/or outdoors.

Suitable applications for the device of this invention can be any indoor and/or outdoor architectural applications including, but not limited to, being part of a building façade, roofs, sun-canopies, sun shades, windows, and/or indoor ceilings, indoor walls, or indoor floors. In these applications, produced oxygen can be used inside the building and/or the CO₂ gas provided to the chambers can come from inside and/or outside the building. Thermal insulation can also be provided to these buildings by the invention.

Suitable applications for the device of this invention can be together with any lighting systems and/or lighting fixtures, including, but not limited to, interior lighting systems such as ceiling, ground, wall, desk, suspended, technical, decorative, outdoor, industrial machinery lighting, vehicle lighting, street lighting, or advertising lighting fixtures.

In such applications, the artificial light source provided from the lighting system can provide most of the light needed by the microorganisms to photosynthesise, and the produced oxygen can be used inside the building and/or the CO₂ can be absorbed from inside and/or outside the building.

Additional suitable applications for the device of this invention can be intensive biomass production applications, including, but not limited to, outdoor intensive biomass production plants using mostly natural light sources, indoor intensive biomass production plants using artificial light sources and/or natural light sources, such as in greenhouses. The biomass can contain food ingredients and/or additives and/or can be used as a protein source for human or animal consumption, or for plant or other fertilising purposes. Further suitable applications for the device of this invention can be together with infrastructures, including, but not limited to, urban infrastructures, motorways, bridges, industrial infrastructures, cooling towers, highways, underground infrastructures, traffic sound barriers, silos, water towers, or hangars.

Other suitable applications for the device of this invention can be in combination with waste treatment plants, including, but not limited to, waste water treatment plants, municipal waste water treatment plants, sewage anaerobic digestion treatments, manure anaerobic digestion treatments, anaerobic digesters or incinerators.

The device of this invention can remove pollutants and/or nutrients (such as nitrates and phosphates) directly from waste water streams which can be diverted inside the units. This is favourable in waste water treatment applications and building/industrial applications where a partial and/or pre-treatment of water is demanded. Water containing contaminants that are toxic to the microorganisms within the device of the invention should in such embodiments be treated to remove these contaminants prior to being introduced into the device.

The device of this invention can be installed on or near to any kind of industrial, agricultural, farming, intensive farming (such as intensive fish farming), manufacture, refinery and/or energy production processes which can supply some or all of the gases for use within the gas chambers of the device.

The device of this invention can be installed inside any industrial machinery and/or vehicle where the chamber can be substantially composed by their body parts and where the device is used to produce biomass, and/or remove the carbon dioxide from the effluent gases produced by the industrial machinery and/or vehicles.

The device of the invention is exemplified by, but in no way limited to, the following arrangements.

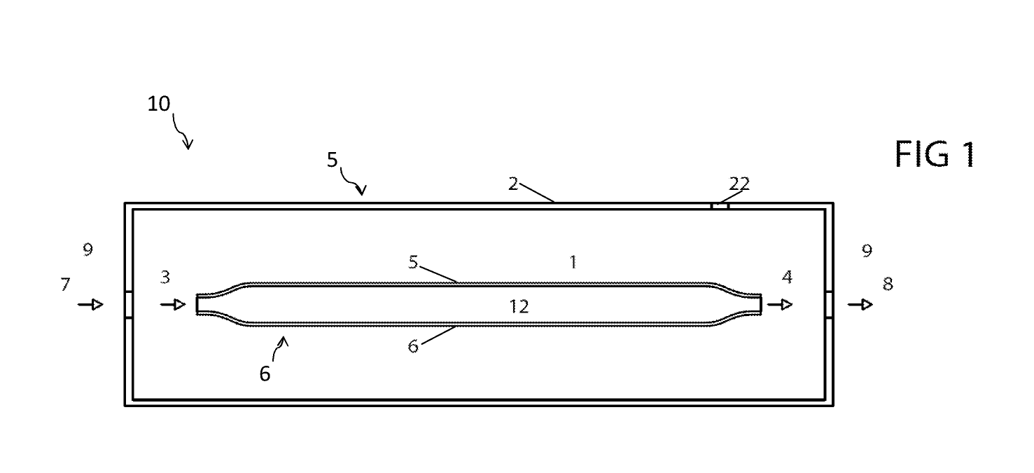

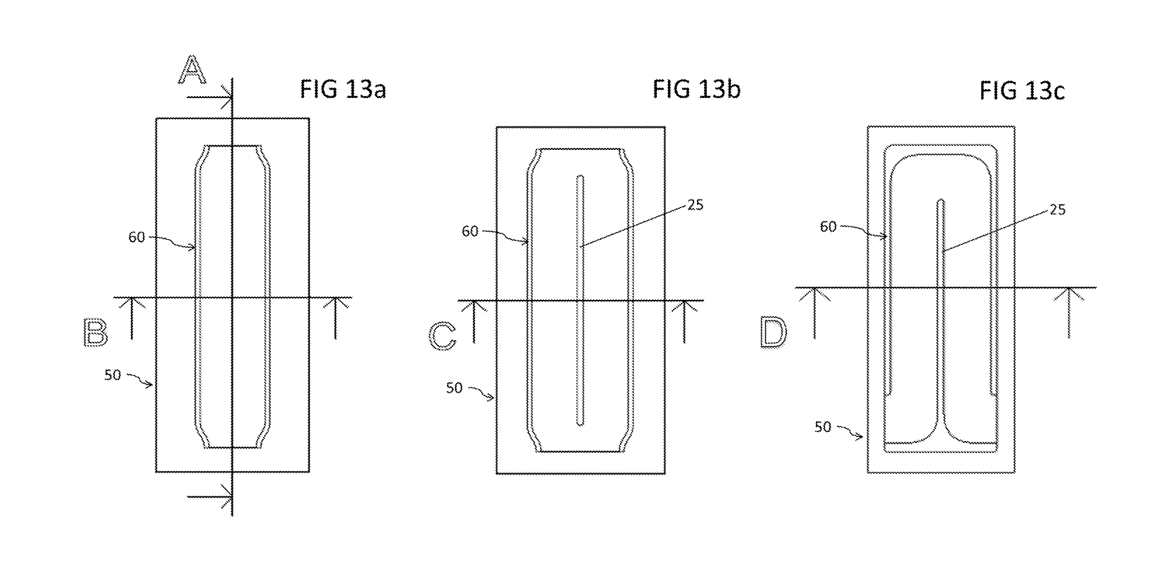

FIG. 1 shows a cross-section (see Section A of FIG. 13a) of a device according to an embodiment of the invention (100), comprising a linear PBR (60) comprising an inlet (3) and outlet (4) located on opposite sides, and outer layers (5, 6), one or both of which is permeable to gases, and liquid media comprising a photosynthetic microorganism (12) contained within the PBR. The PBR is surrounded on substantially all sides by an atmosphere (1) defined by its enclosure within a chamber (50) which comprises walls (2), an inlet (8) and an outlet (7). The chamber (50) and chamber walls (2) separate the atmosphere (1) from the outside atmosphere (9). In some embodiments the chamber further comprises a chamber valve (22) for the removal of gas from the atmosphere (1).

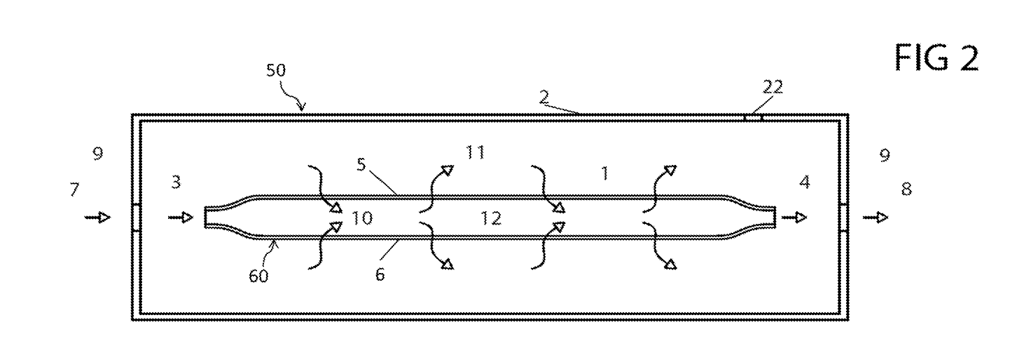

FIG. 2 shows the transfer of gases (10) from the atmosphere (1) to the PBR contents (12) and also (11) from the PBR contents to the atmosphere (1).

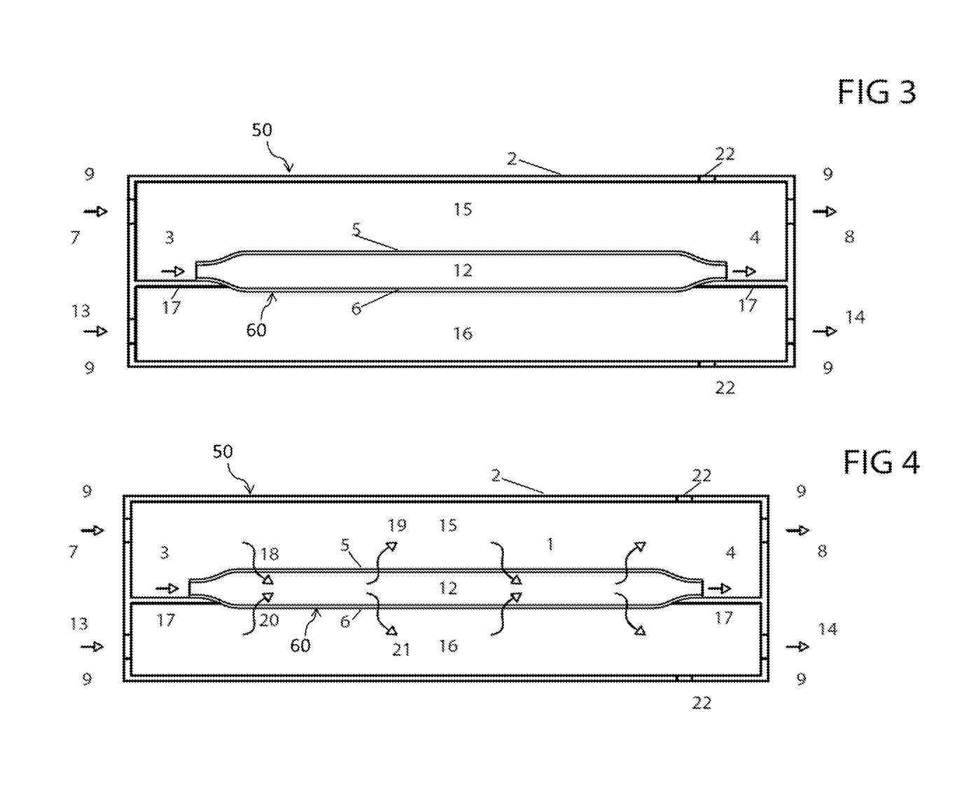

FIG. 3 shows a cross-section of a device according to another embodiment of the invention wherein the chamber (50) is separated into two sections by a dividing wall (17), with a first section comprising an inlet (7) and outlet (8) and an atmosphere (15) and a second section comprising an inlet (13) and outlet (14) and an atmosphere (16).

FIG. 4 shows the transfer of gases between the PBR (60) and the atmospheres of the chambers (15, 16), with transfer shown from the atmospheres to the PBR (18, 20) and from the PBR to the atmospheres (19, 21).

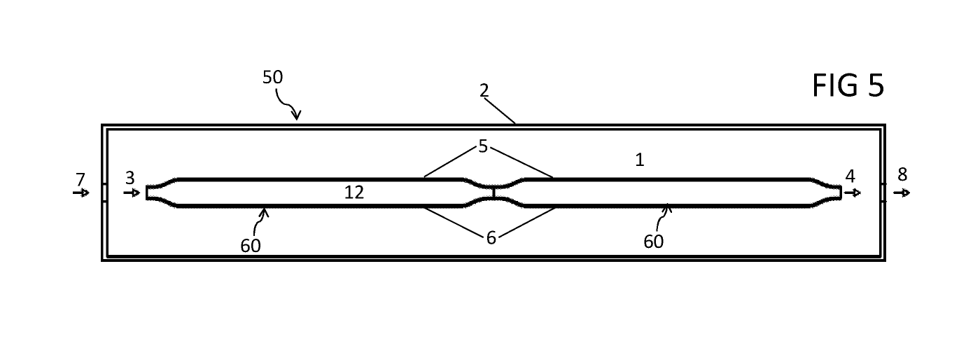

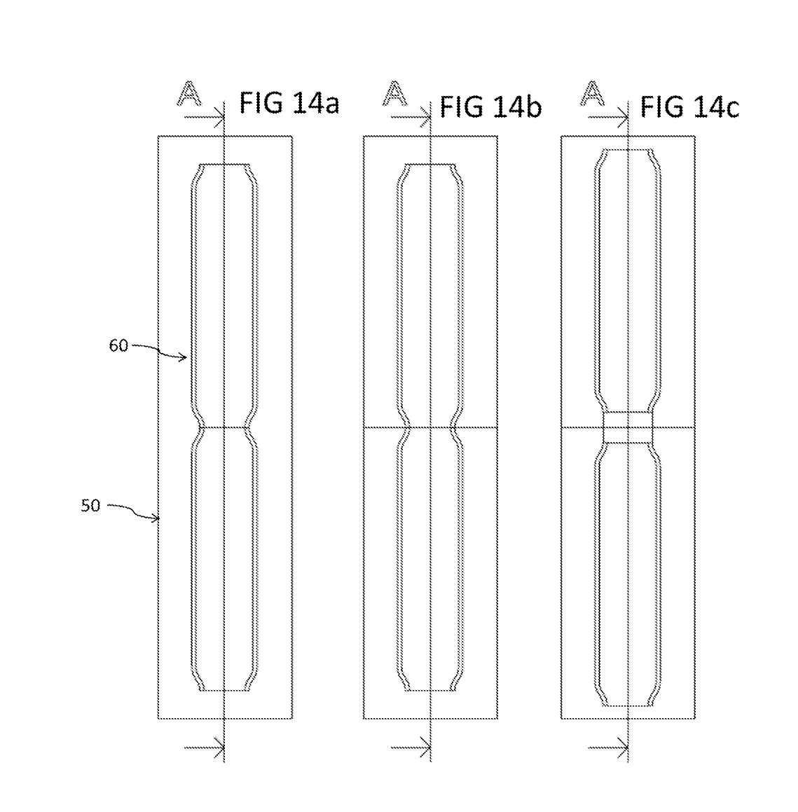

FIG. 5 shows a cross-section (see Section A of FIG. 14a) of an arrangement according to another embodiment of the invention wherein two PBRs (60) are directly connected in series such that their liquid media (12) is in fluid communication, and the PBRs are contained within a single chamber (50). In some embodiments more PBRs may be connected within a single chamber.

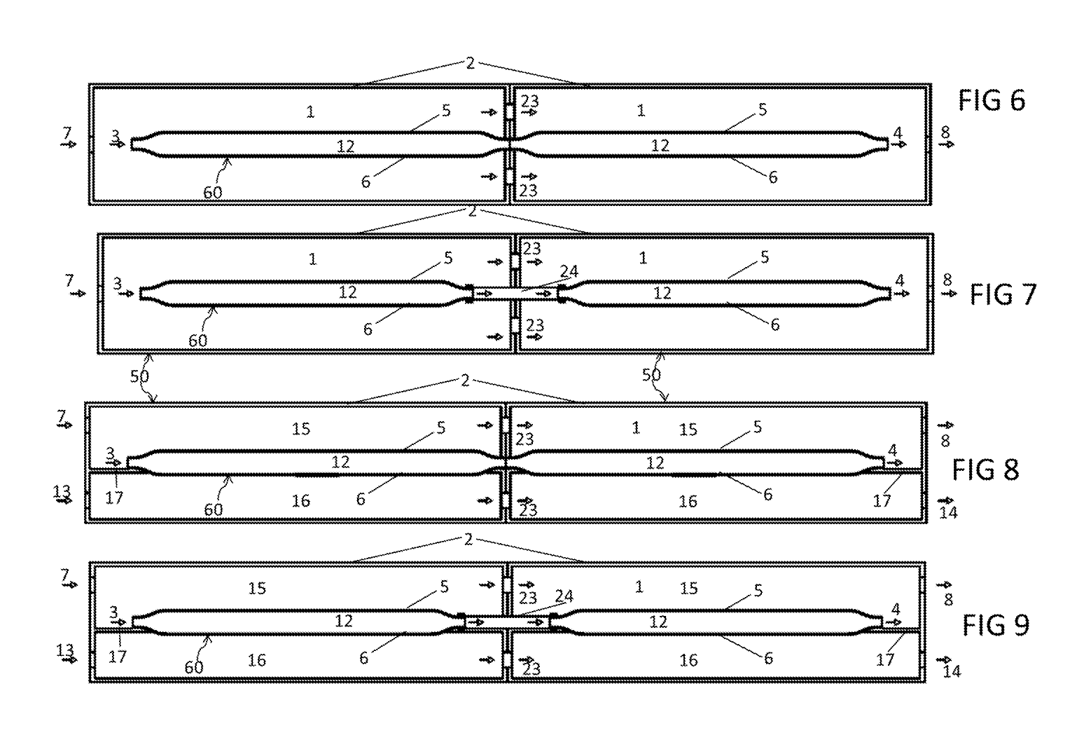

FIGS. 6 and 7 show cross sections (see Section A of FIGS. 14b and 14c) of an arrangement according to another embodiment of the invention wherein two PBRs (60) are directly connected in series, wherein each PBR (60) is contained within a chamber (50). The atmospheres (1) of the chambers (50) are in fluid communication with each other through apertures (23) in the chamber walls (2). The PBRs may be connected via a conduit (24).

FIGS. 8 and 9 show cross sections (see Section A of FIGS. 14b and 14c) of an arrangement according to another embodiment of the invention wherein two PBRs (60) are directly connected in series, and are each contained within a chamber (50). The chambers (50) are each separated into two sections and the atmospheres (15) of each first section are in fluid communication, and the atmospheres (16) of each second section are also in fluid communication.

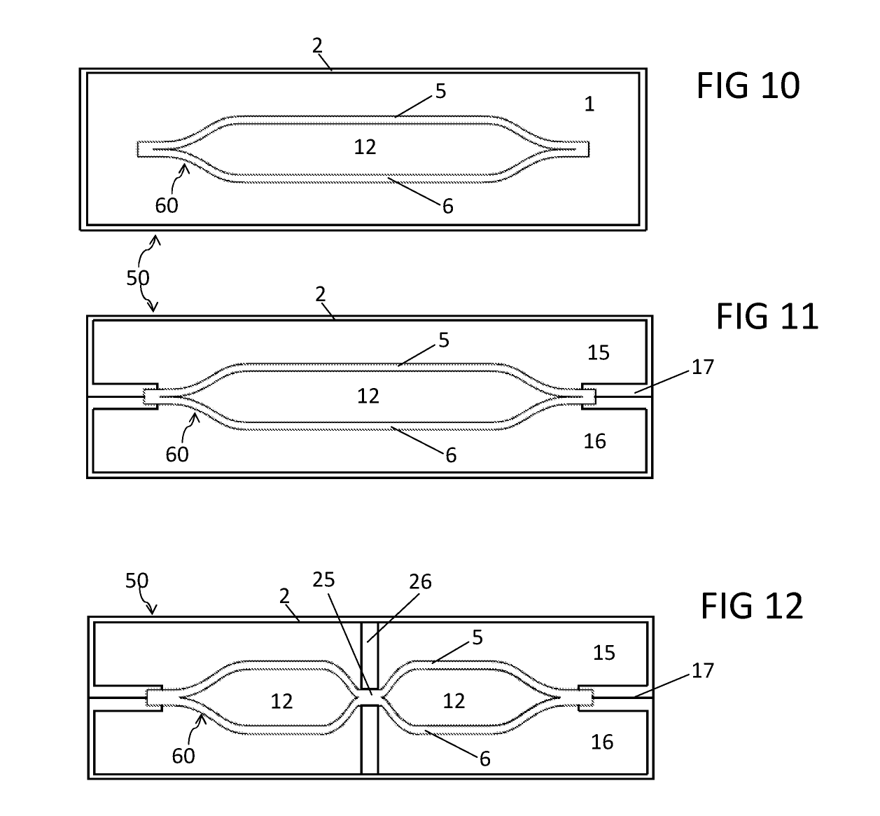

FIGS. 10 to 12 show alternative cross sections of devices according to embodiments of the invention. FIG. 10 (Section B of FIG. 13a) shows a PBR (60) contained within a chamber (50) FIG. 12 (Section C of FIG. 13b) further shows a central flow control structure (25) creating a bifurcated channel, and a support structure (26) maintaining the PBR (60) substantially in the centre of the chamber (50).

FIGS. 13a and 13b show planar sections A, B and C through representations of the device according to the above arrangements. FIG. 13c shows planar section D through representations of the device according to arrangements where the liquid media follows a sinuous or tortuous path.

FIGS. 14a, b and c show the planar section A through a representation of the device according to an embodiment of the invention.

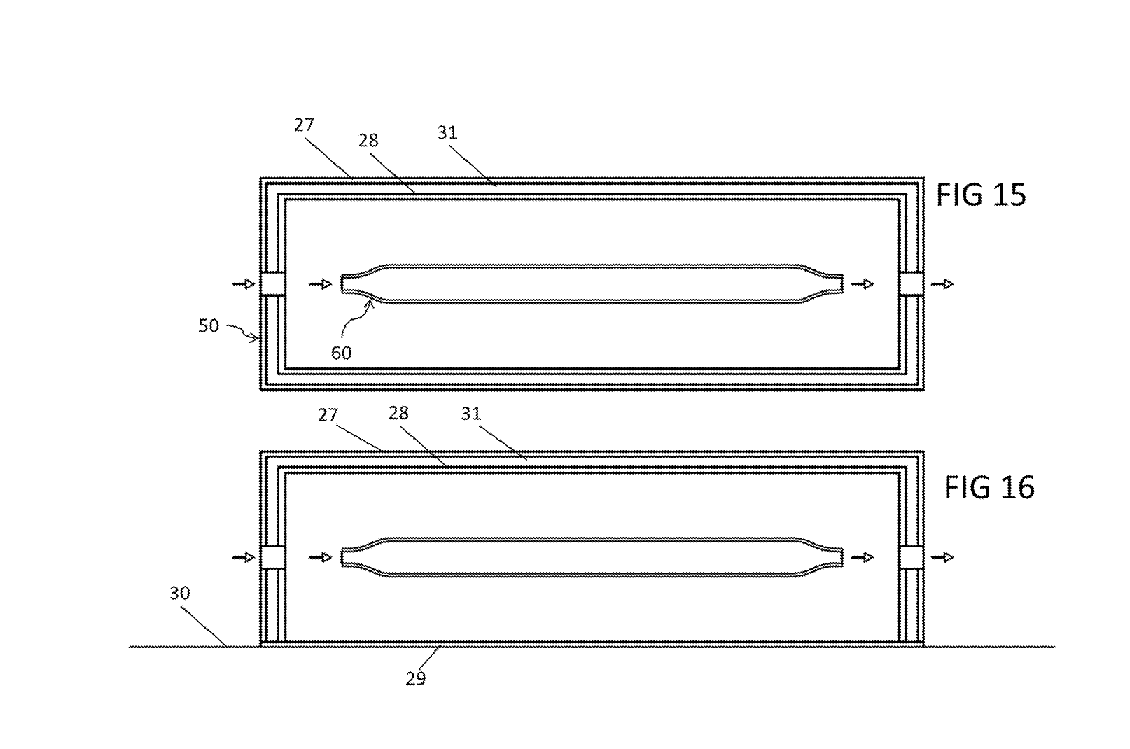

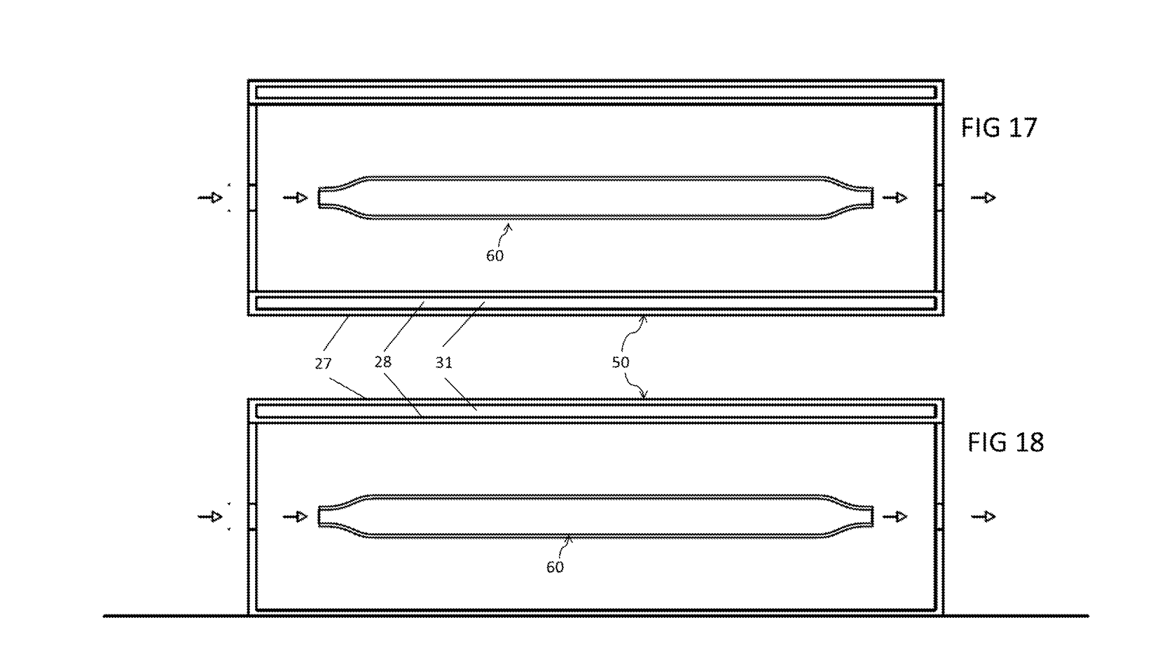

FIGS. 15 to 18 show cross-sections of devices according to embodiments of the invention having a linear photobioreactor (60) enclosed within a chamber (50), wherein one or more of the walls of the chamber are made up of two layers, an inner layer (28) and outer layer (27) with an intervening space (31). The lower wall may be positioned against a surface (30).

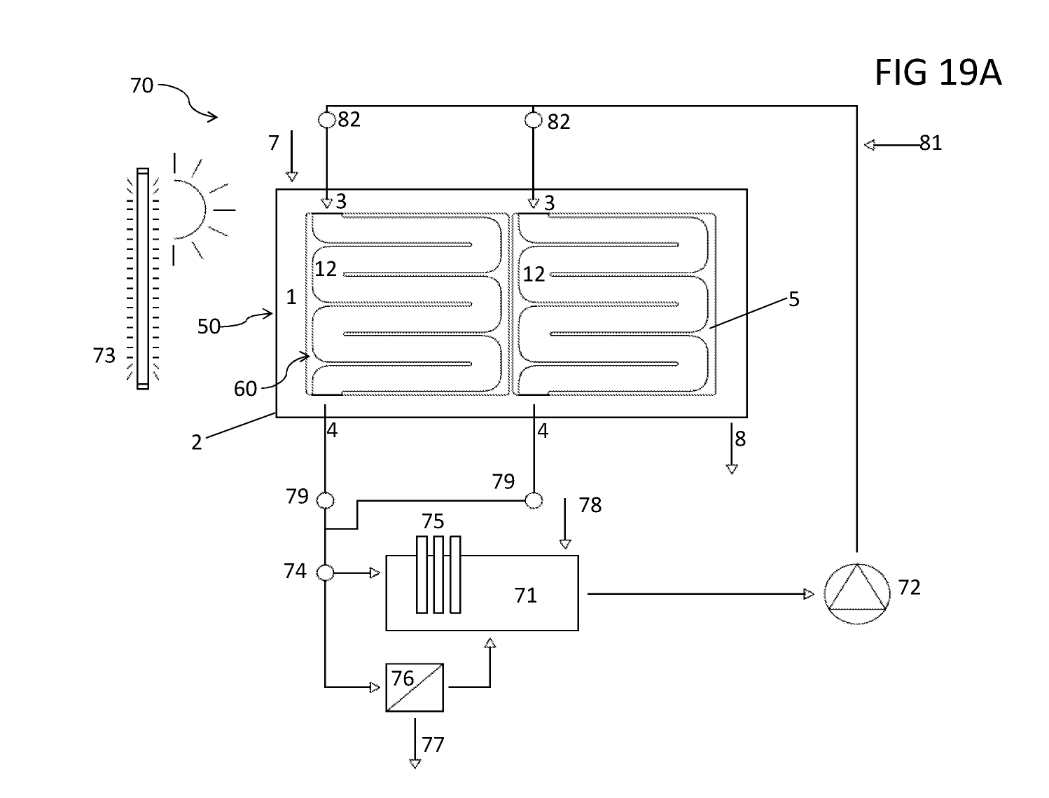

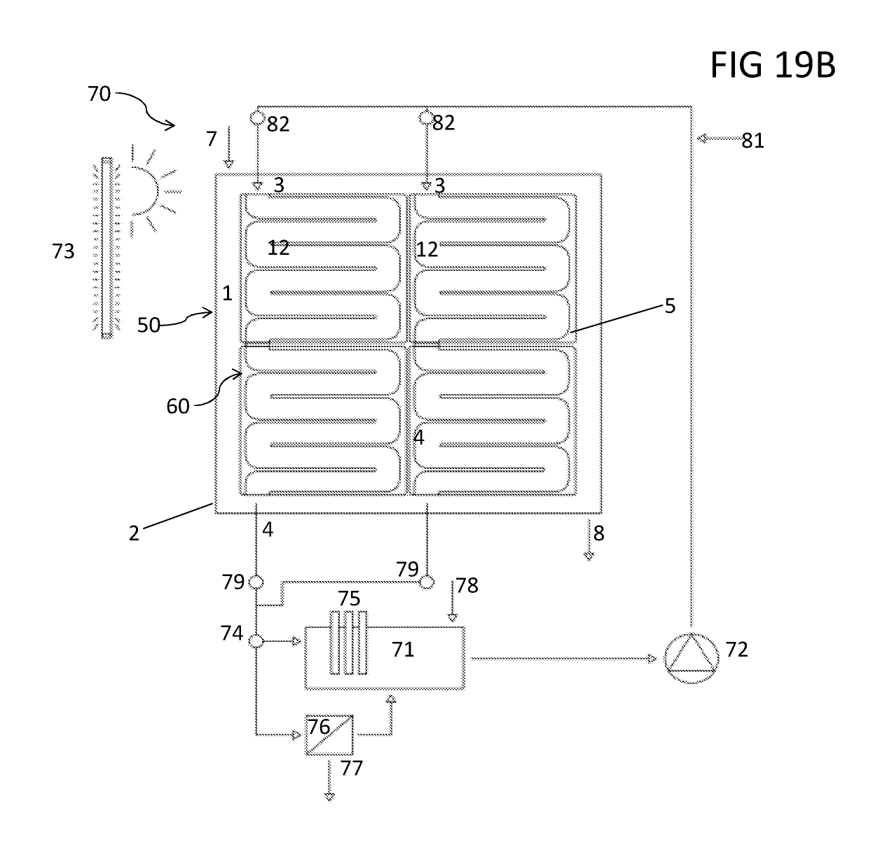

FIG. 19a shows a suitable system (70) of one embodiment of the invention, comprising multiple PBRs. The liquid media (12) comprising a photosynthetic microorganism in a reservoir (71) is conveyed by a pump (72) into a rectangular PBR through the inlet (3). The PBR is enclosed within a chamber which also encloses an atmosphere (1), controlled by gas movement through an inlet (7) and outlet (8). The liquid media passes along a tortuous path through the PBR where light from an artificial light source (73) or natural light source reaches the microorganisms in the liquid media (12) stimulating photosynthesis, meanwhile gas transfer between the liquid media in the unit (12) and the atmosphere (1) occurs through the membrane layers of the unit substantially as shown, for example, in FIG. 2. The liquid leaves the unit through the outlet (4) and reaches a 3-way valve (74) which directs the liquid media back into the reservoir (71), closing the circuit. Sensors (75) in the reservoir (71) measure the values of microorganisms culturing parameters and send outputs to the computers which then control operations of the auxiliary system's components, such as pumps, valves, artificial light systems, temperature control systems, biomass-separators. Computers also control supply of gases to the chamber atmosphere (1) through the inlet (7) and gas removal through the outlet (8). FIG. 19b shows a similar system, with two PBRs connected in series.

When the biomass concentration in the liquid media reaches the desired level, the 3-way valve (74) directs the flow into the biomass-separator system (76) which separates the biomass from part of the liquid media, the isolated biomass proceeds into a receptacle (77) for additional processing, while the liquid media is directed back into the reservoir (71). This action of directing the flow into the biomass-separator can be performed periodically and for a predetermined period of time before the valve (74) changes the flow path into the reservoir (71) again. This timing can be optimised with respect to each application, the microorganism used, the surrounding environment and location of the device. Alternatively the 3-way valve (74) can regulate the flow to the reservoir (71) and the biomass separation system (76) to enable a continuous harvest of biomass while allowing for dynamic control of the quantity of biomass removed from the system at a given time. For example the valve (74) can deliver between 0% and 100% of all the liquid media that pass through the valve to the biomass separation system (76).

Nutrients can be periodically inserted (78) in the system directly into the reservoir (71). Water and/or microorganisms in liquid media, or cleaning fluid, can be similarly introduced.

All sorts of other system components can be utilised, as example a controllable pressure valve or pressure regulator (79) can be placed in the system, in this example the pressure valve can control the volumetric change of the unit through the effects of changes in the liquid pressure. Some valves (82) can control the flow rate into the units. Supplementary air and/or air enriched with carbon dioxide and/or other gases can optionally be introduced (81) in the main conduit if required, in addition to the gas supply to the chamber. Air vents can be installed in the conduits to remove air that can accidentally enters the hydraulic system, for example during installation of the system, and are typically located in the highest location of the system to facilitate the expulsion of undesirable air.

A cleaning procedure can be actuated to clean and/or sterilise the unit and/or the conduits and/or the water tank and/or all the auxiliary system and/or the gas chamber. The cleaning procedure can be performed by using steam or heated air or water vapour as a cleaning medium. A “cleaning fluid” can be made of any compound the skilled person will know. It may comprise ethanol, water, hydrogen peroxide (H₂O₂), salty water, detergents, bleach, surfactants, alkali or any other suitable cleaning composition. The cleaning liquid can enter the system through specific conduits in any point of the system and can exit at any point of the system to permit cleaning in specific locations only, if desired, instead of cleaning the entire system.

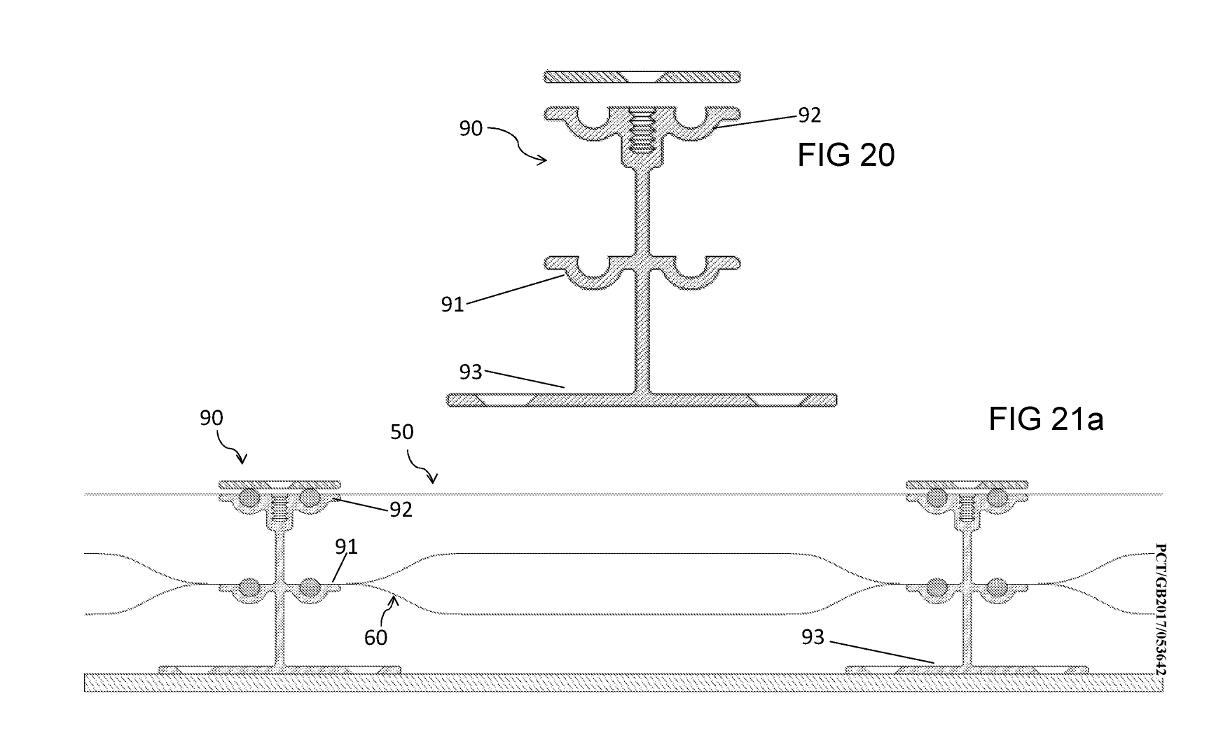

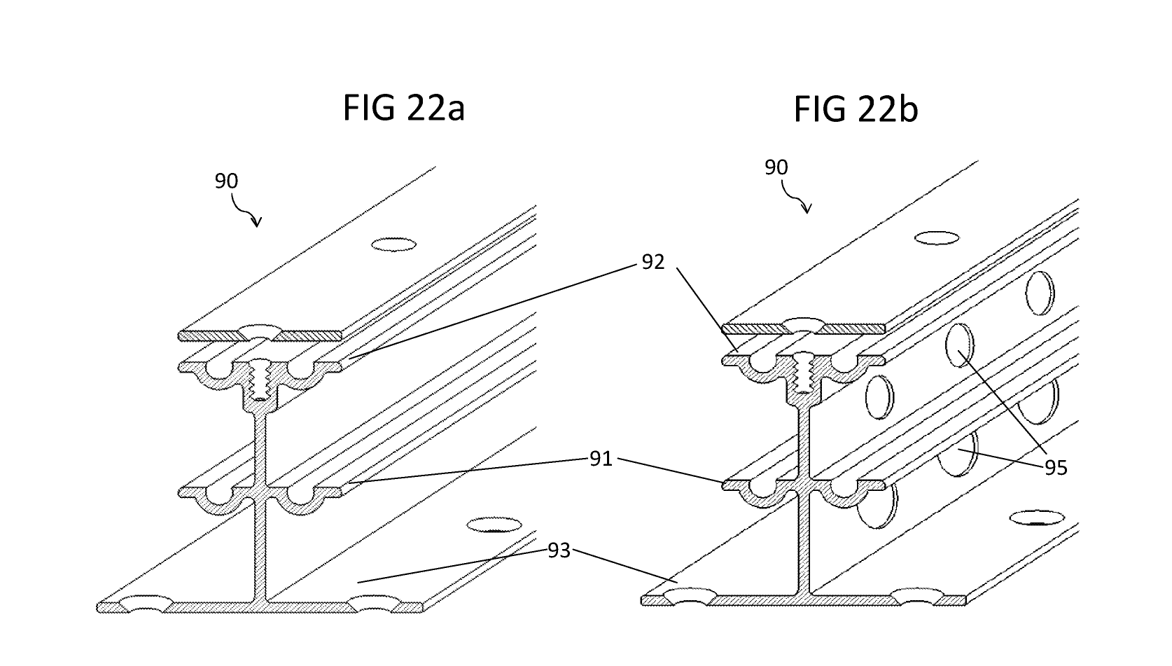

FIGS. 20 to 23 show that the chamber assembly may comprise a support structure (90) which may be comprised of a metal and/or plastic structure, for example an extruded structure, that extends linearly (following desired PBR array) on two sides, The extruded structure may function as the structural support for the membrane PBR, the upper and the bottom surface. The extruded structure may comprise housing mechanisms or fittings (91, 92, 93) to fix and/or hold in place the PBRs (91), the upper walls of the chamber (92) and the lower walls of the chamber (93). The ends on the modules can be closed by other support structure elements in order to create a closed chamber. The walls of the extruded structure (see FIG. 22b) may comprise holes (95) which enable gas to travel from one chamber section to another especially in embodiments which comprise an array of multiple chambers.

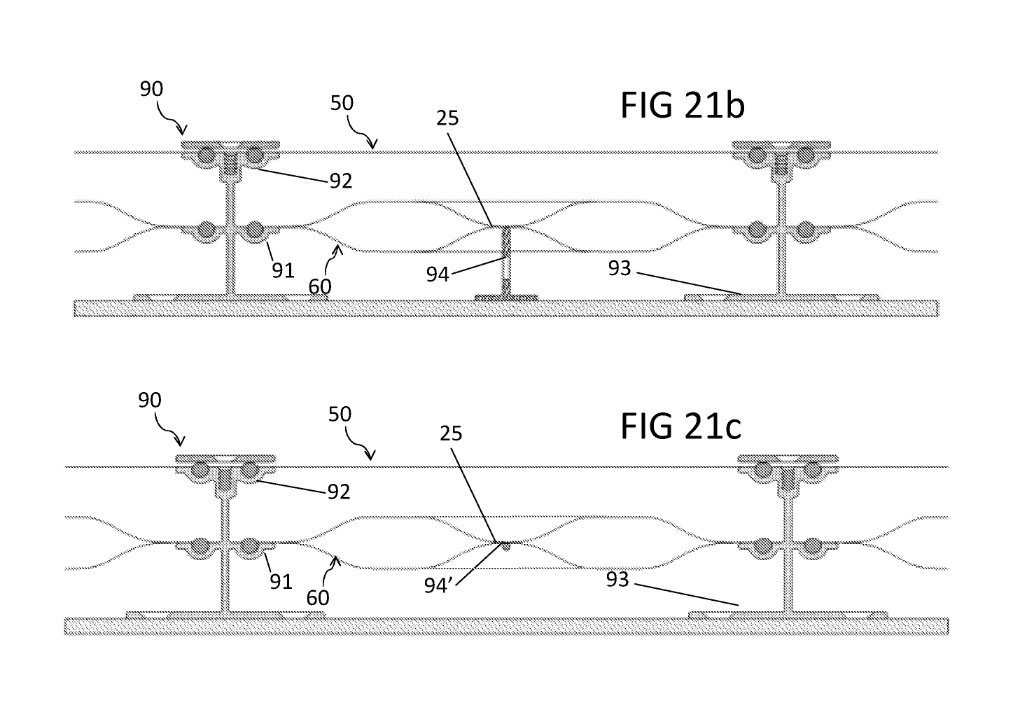

FIGS. 21b and 21c show additional configurations for supporting the PBR within the chamber assembly, by the addition of a suspension member which may be a fin (94) mounted on the lower wall of the chamber or a cord (94′) that is suspended between side walls. This suspension member supports the centre of the PBR, to prevent sagging and reduce the possibility of damage or strain on the connections of the PBR to the support structure.

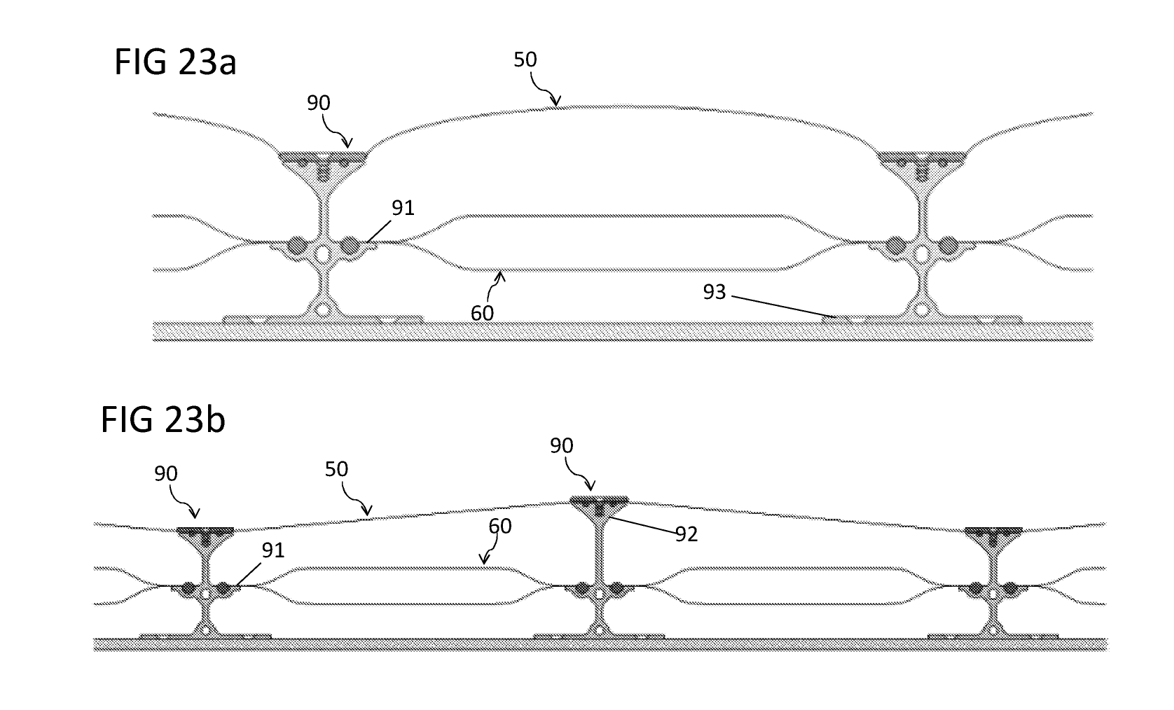

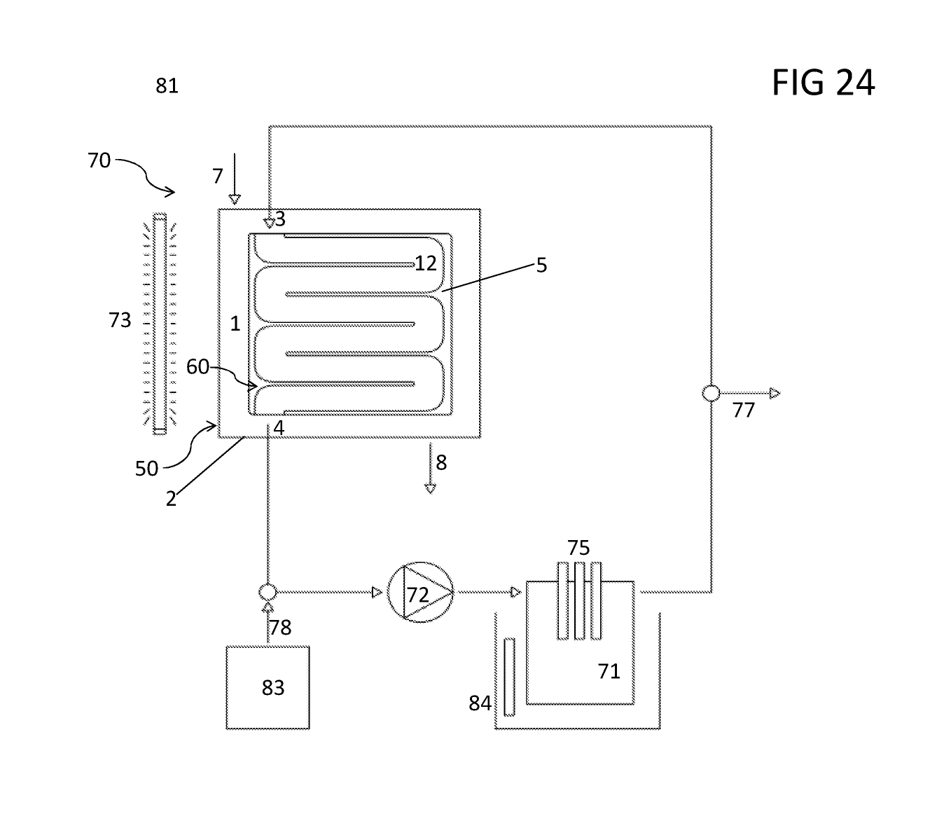

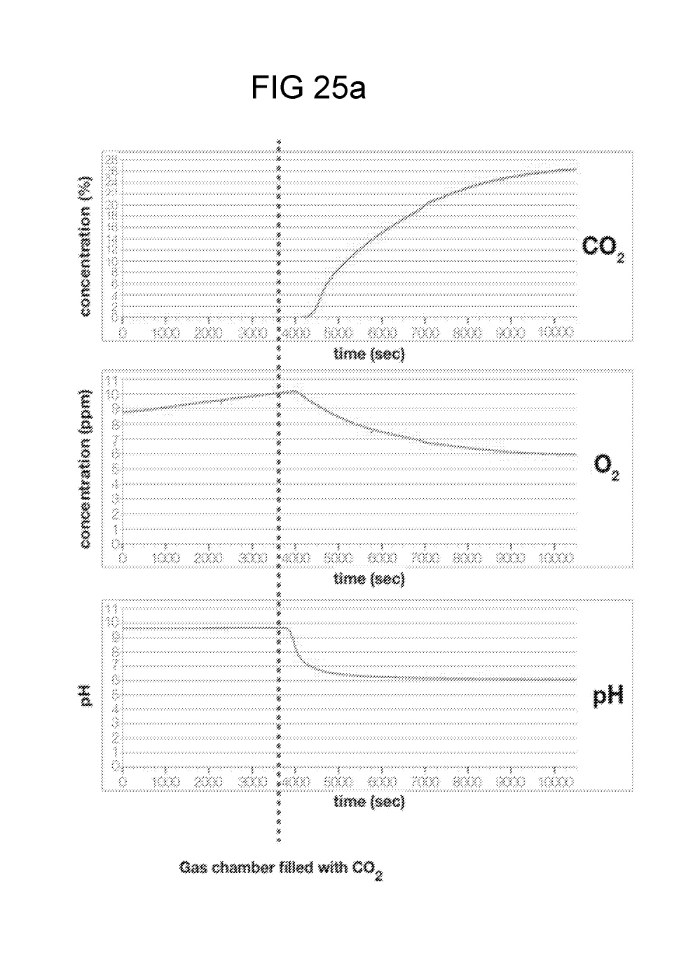

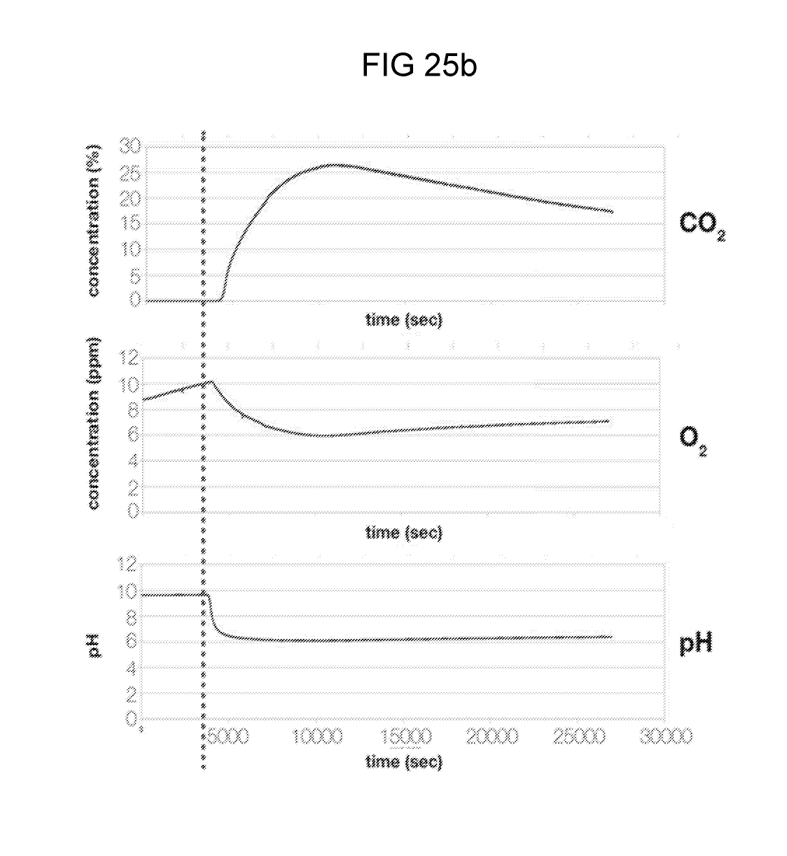

FIGS. 23a and 23b show embodiments of the invention which are adapted to prevent the collection of water or other substances on horizontal surfaces of the apparatus, and so reduce light interference. In FIG. 23a the upper wall of the chamber has a rounded convex shape, so that water or other substances run off this surface. FIG. 23b has support structures (90) of differing heights, such that the upper wall of the chamber is tilted relative to the horizontal, again encouraging runoff. Another advantage of such embodiments is that condensation on the inside of the upper wall is encouraged to run away from positions directly above the PBR.

An exemplary configuration of the invention is as follows. A breathable membrane PBR made of two layers of a transparent polysiloxane compound gas permeable membrane of thickness 50-100 μm.

The PBR is located within a chamber assembly. The chamber assembly is made of a steel chassis (box) with an opening window on the superior surface exposed to light. This opening window is glazed with a transparent ETFE layer (thickness in the range of 100-500 μm).

The PBR is stretched and fixed on the support chassis by eyelets on the border of the PBR being fixed to horizontal members welded onto the chassis. A holding structure on the bottom inside surface of the chassis maintains the position of the PBR at the centre of the gas-chamber. The holding structures contact the PBR in the location where the layers of the PBR are fused to create flow control structures, to avoid the holding structures interfering with gas transfer through the PBR membranes.

In this way the majority of the PBR surface both on the top and bottom is exposed to the atmosphere of the gas-chamber and permits circulation of the atmosphere thereabout.

The PBR has inlets and outlets for the contained liquid media and is connected to an auxiliary system comprising a water tank which comprises sensors for pH, dissolved O₂ and CO₂, temperature, and turbidity, and further comprising a peristaltic pump and water heating system.

The chamber assembly is substantially air-tight. It possesses an inlet for feed gas and outlet for effluent gas, both controlled by solenoid valves actuation of which are under the control of a programmable logic controller (PLC). The inlet is further connected to a CO₂ canister and/or to a Nitrogen gas canister.

CO₂ is pumped into the gas chamber, with the outlet valve open in order to allow for the removal of the atmospheric air previously contained in the gas chamber. CO₂ is pumped without increasing atmospheric pressure inside gas chamber.

The invention is further exemplified by reference to the following non-limiting examples.

### EXAMPLES

### Example 1

An experimental apparatus was constructed to demonstrate a system according to an embodiment of the present invention. In particular, the apparatus demonstrates that supplying CO₂ gas into the gaseous atmosphere of a chamber containing a PBR of the type described herein results in an increase in CO₂ concentration, along with a decrease in O₂ concentration and pH within the liquid media comprised within the PBR. This further indicates that efficient O₂ and CO₂ gas transfer occurs through the membrane layers of the PBR unit filled with a liquid media that comprises a photosynthetic microorganism culture.

The case study set-up is represented by a simplified schematic in FIG. 24. This set up defines a system according to one embodiment of the present invention. With reference to FIG. 24, the majority of the features shown in this schematic are the same as those found in FIGS. 19a and 19b. In addition, a tank (83) is shown, which contains a reserve of liquid media, and the reservoir (71) is heated by a water bath (84).

A PBR unit (5) was constructed from two polysiloxane membrane layers, 100 microns thick, having a permeability coefficient (ISO 15105-1) of O₂ equal to approximately 400 Barrers, of CO₂ equal to approximately 2100 Barrers and nitrogen equal to approximately 200. The PBR measured approximately 450 by 450 mm and was constructed by joining two membrane layers using VVB adt-x silicone adhesive in between the layers and heat pressing them to create a continuous channel defining a tortuous path.

The PBR was filled to its normal operating capacity with liquid media containing BG11 cyanobacteria freshwater medium and Synechocystis sp. culture PCC6803. The system is air tight, therefore gas exchange between the liquid media within the PBR and the atmosphere within the surrounding chamber occurs solely through the polysiloxane membrane layers of the unit (5). Gas can be vented from the chamber via a valve (8) to control the pressure and gas mix of the atmosphere.

The chamber (50) was constructed from a steel chassis (box) with an opening window on the superior surface exposed to light. This opening window is glazed with a transparent ETFE layer approximately 200 μm thick. The PBR was stretched and fixed on the support chassis by eyelets on the border of the PBR being fixed to horizontal members welded onto the chassis. The PBR was supported within the chamber by acrylic 1.5 mm thick holding structures rested perpendicularly on the floor of the chamber. The holding structures contacted the PBR in the location where the layers of the PBR were fused to create flow control structures, in order to avoid the presence of the holding structures interfering with gas transfer through the PBR membranes, and to avoid puncturing or cutting the PBR. At the beginning of the experiment the chamber was filled (once) with atmospheric air. During the experiment, a CO₂ flush was conducted to replace the air atmosphere within the chamber.

Pressurised CO₂ was supplied from a cylinder from BOC and introduced into the chamber via an inlet valve (7) with air vented from an outlet valve (8).

The reservoir (71) is designed to be air tight and to accommodate the sensors (75). The sensors (75) used for this case study were:
1. an optical dissolved O₂ sensor “InPro 6860 i” from Mettler Toledo,
2. a Dissolved CO₂ sensor “InPro 5000 I”, from Mettler Toledo,
3. a pH sensor from Hannah Instruments,
4. a temperature sensor IFM Efector TM4431 PT100
5. a pressure transmitter with ceramic measuring cell IFM Efector PA9028

Illumination of the system was provided by a Lightwave T5 Propagation Grow Light system fitted with 8×4 ft T5 fluorescent tubes using dimmable drivers.

The liquid media temperature was maintained at approximately 29° C. (±2° C.), the liquid media temperature was maintained by a heated secondary water bath which surrounded the main reservoir (71). The liquid media was pumped throughout the system by a peristaltic pump (VerderFlex Steptronic EZ pump) (72). One 3-way pinch solenoid valve (SIRAI S307) can diverge the liquid media coming from the PBR out of the system into a receptacle for biomass harvesting and further liquid media sampling (i.e. culture total density/biomass weighting) when needed, while another 3-way pinch valve enables the insertion into the system of new liquid media containing BG11 medium from an auxiliary water tank. Data related to dissolved gas concentration level and pH in the liquid media was recorded.

The concentration of O₂ was seen to rise by approximately 1 ppm over the early stage of the experiment, this was believed to be an artefact associated with the start-up of the system; the system was run for over 50 minutes before the introduction of CO₂ to attempt to bring the system to equilibrium. In another experiment, with a PBR in a chamber filled with atmospheric air, but maintained at a lower temperature, O₂ concentration did not rise significantly and was stable for at least 15 minutes, as shown in Table 1.

TABLE 1

| Time | | | | |
|---|---|---|---|---|
| (minutes) | pH | Temperature (° C.) | O₂ (ppm) | CO₂ (% concentration) |
| 0 | 9.4 | 22.9 | 8.5 | 0 |
| 5 | 9.3 | 22.9 | 8.5 | 0 |
| 10 | 9.4 | 22.9 | 8.6 | 0 |
| 15 | 9.4 | 22.9 | 8.6 | 0 |
| 20 | 9.4 | 23.2 | 8.6 | 0 |
| 25 | 9.4 | 23.2 | 8.6 | 0 |

As shown in the graph illustrated in FIGS. 25a and 25b (these graphs depict the same experiment over different time scales, as shown), at approximately 3600 seconds, as denoted by the vertical dashed line, the chamber was flushed with 100% CO₂ for approximately 120 seconds, until the air previously within had been displaced. As shown in FIG. 23a, the pH of the liquid media declined over this period, indicating the effect of an increased CO₂ concentration on pH. When the pH reached a value of approximately 7.5, to represent direct effects on controlling the internal chamber atmosphere, the chamber was opened to the atmosphere via a vent, without flushing the internal atmosphere, and the levels of CO₂ were allowed to fall gradually due to influx of the external air atmosphere.

As shown in the same graphs, the concentration of dissolved CO₂ (indicated in % of total concentration) within the liquid media of the PBR increased after the CO₂ flushing, and the concentration of dissolved O₂ (indicated in ppm) declined at the same time, both changes approaching a plateau at around 10000 seconds. This indicated gas exchange taking place across the PBR membrane between the liquid media and the CO₂-enriched atmosphere within the chamber.

Approximately 8000 seconds after the 120 second supply of CO₂, the concentration of dissolved CO₂ was seen to decline, and that of O₂ to increase showing that reversal of the effects of CO₂ supply is possible, by the action either of microorganism processes or the fall in CO₂ concentration within the chamber atmosphere due to venting of CO₂ to the atmosphere.

### Example 2

To show that organism growth and replication occurs inside the device, in another similar experiment in a device according to the invention, samples of liquid media were taken from the system at different time intervals, and dry weight measurement was performed, to understand the total biomass density and growth rate. As shown by the table below the total biomass grew by 0.8 g/l over just over 8 hours, an increase of over 40% over this time frame.

TABLE 2

| Time (hours:minutes) | Dry Biomass weight |
|---|---|
| 00:00 | 1.9 g/l |
| 05:11 | 2.4 g/l |
| 08:15 | 2.7 g/l |
| 24 hours (approx.) | 2.9 g/l |

Although particular embodiments of the invention have been disclosed herein in detail, this has been done by way of example and for the purposes of illustration only. The aforementioned embodiments are not intended to be limiting with respect to the scope of the appended claims, which follow. It is contemplated by the inventors that various substitutions, alterations, and modifications may be made to the invention without departing from the spirit and scope of the invention as defined by the claims.

1. A device for the production of biomass, the device comprising:
a membrane photobioreactor (PBR), the PBR comprising a liquid medium, at least one photosynthetic microorganism, and at least one outer membrane layer, wherein the membrane layer is comprised of a material that is permeable to transfer of a gas across the membrane layer, and wherein the gas comprises carbon dioxide; and
a chamber defining a gaseous atmosphere enclosed within;
wherein the PBR is located inside the chamber; and
a control system which controls the composition of the atmosphere within the chamber;
wherein the permeability coefficient of carbon dioxide (CO2) through the membrane layer of the PBR is not less than about 1000 Barrer, such that gas transfer occurs across the membrane layer of the PBR, between the PBR and the atmosphere comprised within the chamber.

2. The device of claim 1, wherein the chamber is comprised of a plurality of walls and at least one wall, or a portion thereof, permits the transmission therethrough of visible light into the interior of the chamber.

3. The device of claim 1, wherein the chamber comprises a source of illumination.

4. The device of claim 1, wherein the walls of the chamber are substantially rigid.

5. The device according to claim 1, wherein the walls of the chamber comprise ethylene tetrafluoroethylene (ETFE).

6. The device of claim 1, wherein the membrane layer of the PBR is substantially transparent.

7. The device of claim 1, wherein the membrane layer of the PBR comprises polysiloxane.

8. The device of claim 1, wherein the permeability coefficient of oxygen through the membrane layer of the PBR is selected from: not less than about at least 100, suitably not less than at least 200, at least 300, at least 400, at least 500, at least 650, at least 750, suitably at least 820 Barrer.

9. The device of claim 1, wherein the permeability coefficient of carbon dioxide through the membrane layer of the PBR is selected from: not less than at least 1500, at least 2000, at least 2200, at least 2500, at least 2800, at least 2900, at least 3000, at least 3100, at least 3200, at least 3300, at least 3400, at least 3500, at least 3600, at least 3700, at least 3800, suitably at least 3820 Barrer.

10. The device of claim 1, wherein the PBR is substantially surrounded on all sides by the atmosphere within the gas impermeable chamber.

11. The device of claim 1, comprising multiple PBRs located inside the chamber, wherein the liquid media of the PBRs are in fluid communication.

12. The device of claim 1, wherein the at least one photosynthetic microorganism is selected from one or more of the group consisting of: Haematococcus sp., Haematococcus pluvialis, Chlorella sp., Chlorella autofraphica, Chlorella vulgaris, Scenedesmus sp., Synechococcus sp., Synechococcus elongatus, Synechocystis sp., Arthrospira sp., Arthrospira platensis, Arthrospira maxima, Spirulina sp., Chlamydomonas sp., Chlamydomonas reinhardtii, Dysmorphococcus sp., Geitlerinema sp., Lyngbya sp., Chroococcidiopsis sp., Calothrix sp., Cyanothece sp., Oscillatoria sp., Gloeothece sp., Microcoleus sp., Microcystis sp., Nostoc sp., Nannochloropsis sp., Anabaena sp., Phaeodactylum sp., Phaeodactylum tricornutum, Dunaliella sp., Dunaliella salina.

13. The device of claim 1, wherein the chamber is divided into two or more sections to provide at least a first chamber section and a second chamber section.

14. The device of claim 1, wherein the control system is configured to introduce a CO₂-rich gas into the chamber.

15. The device of claim 1, wherein the control system is configured to introduce an O₂-depleted gas into the chamber.

16. The device of claim 1, wherein the control system is configured to introduce an effluent gas from an industrial or combustion source into the chamber.

17. The device of claim 1, wherein the control system is configured to introduce gas into the chamber such that the pressure within the chamber is greater than atmospheric pressure.

18. The device of claim 1, wherein the chamber is substantially gas impermeable.

19.-31. (canceled)

32. The device of claim 7, wherein the membrane layer of the PBR comprises polydimethylsiloxane (PDMS).

33. The device of claim 1, wherein the liquid medium has a pH, and wherein the pH is controlled by the gaseous atmosphere within the chamber.
